(19) United States
(12) Patent Application Publication
(10) Pub. No.: US 2007/0196415 A1
(43) Pub. Date: Aug. 23, 2007

(54) DEPOT COMPOSITIONS WITH MULTIPLE DRUG RELEASE RATE CONTROLS AND USES THEREOF

(76) Inventors: Guohua Chen, Sunnyvale, CA (US); Lothar Walter Kleiner, Los Altos, CA (US); Paul R. Houston, Hayward, CA (US); Jeremy Corwin Wright, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/553,925

(22) Filed: Oct. 27, 2006

Related U.S. Application Data
(63) Continuation-in-part of application No. 10/295,527, filed on Nov. 14, 2002.

(51) Int. Cl. A61F 2/02

(52) U.S. Cl. 424/422

(57) ABSTRACT

Injectable depot compositions with dual mechanisms of release rate control are provided for sustained beneficial agent delivery in a patient. The composition includes bioerodible particles and an injectable depot vehicle containing a bioerodible polymer in an organic solvent, for forming a bioerodible depot implant after administration to the patient. The bioerodible particles are dispersed in the depot vehicle and contain a beneficial agent and a release rate controlling agent retarding the release of the beneficial agent from the bioerodible particles and from the depot implant.

### CROSS-REFERENCE TO RELATED APPLICATION

This application is a continuation-in-part application of and claims the priority benefit of U.S. patent application Ser. No. 10/295,527, which was filed on Nov. 14, 2002 and claimed priority to Provisional Application No. 60/336,307, filed on Nov. 14, 2001. All said prior applications are incorporated by reference herein.

### TECHNICAL FIELD

The present invention relates to a depot composition that can be implanted into a desired location within a patient's body to form an implant, which provides for sustained release of a beneficial agent. The present invention also relates to methods of controlling release of a beneficial agent from a composition and methods of using the depot composition to administer a beneficial agent to a patient.

### BACKGROUND

Biodegradable polymers have been used for many years in medical applications. Illustrative devices composed of the biodegradable polymers include sutures, surgical clips, staples, implants, and drug delivery systems. The majority of these biodegradable polymers have been based upon glycolide, lactide, caprolactone, and copolymers thereof.

With solid implants, the drug delivery system has to be inserted into the body through an incision. These incisions are sometimes larger than desired by the medical profession and occasionally lead to a reluctance of the patients to accept such an implant or drug delivery system. Nonetheless, both biodegradable and non-biodegradable implantable drug delivery systems have been widely used successfully.

One way to avoid the incision needed to implant drug delivery systems is to inject them as small particles, microspheres, or microcapsules. For example, U.S. Pat. No. 5,019,400 describes the preparation of controlled release microspheres via a very low temperature casting process. These materials may or may not contain a drug that can be released into the body. Although these materials can be injected into the body with a syringe, they do not always satisfy the demand for a biodegradable implant. Because they are particulate in nature, they do not form a continuous film or solid implant with the structural integrity needed for certain prostheses. When inserted into certain body cavities such as a mouth, a periodontal pocket, the eye, or the vagina where there is considerable fluid flow, these small particles, microspheres, or microcapsules are poorly retained because of their small size and discontinuous nature. Further, the particles tend to aggregate and thus their behavior is hard to predict. Furthermore, one other major limitation of the microcapsule or small-particle system is their lack of reversibility without extensive surgical intervention. That is, if there are complications after they have been injected, it is considerably more difficult to remove them from the body than with solid implants.

People have developed various drug delivery systems in response to the aforementioned challenges. For instance, U.S. Pat. No. 4,938,763 and its divisional U.S. Pat. No. 5,278,201 relate to a biodegradable polymer for use in providing syringeable, in-situ forming, solid biodegradable implants for animals.

U.S. Pat. No. 5,599,552 describes thermoplastic and thermoset polymer compositions that utilize solvents that are miscible to dispersible in water, such as N-methyl-2-pyrrolidone, resulting in polymer solutions capable of quickly absorbing water from surrounding tissue.

U.S. Pat. No. 5,242,910 describes a sustained release composition for treating periodontal disease. The composition comprises copolymers of lactide and glycolide, triacetin (as a solvent/plasticizer) and an agent providing relief of oral cavity diseases. The composition can take the form of a gel and can be inserted into a periodontal cavity via a syringe using either a needle or a catheter. One illustrative viscosity-controlling agent set forth in one of the examples is polyethylene glycol 400. U.S. Pat. Nos. 5,620,700 and 5,556,905 relate to polymer compositions for injectable implants using solvents and/or plasticizers.

Prior polymer compositions for injectable implants have used solvent/plasticizers that are very or relatively soluble in aqueous body fluids to promote rapid solidification of the polymer at the implant site and promote diffusion of drug from the implant. However, it has now been observed that a serious problem associated with prior polymeric implants utilizing water soluble polymer solvents is the rapid migration of water into the polymer composition when the implant is placed in the body and exposed to aqueous body fluids. That characteristic often results in uncontrolled release of beneficial agent that is manifested by an initial, rapid release of beneficial agent from the polymer composition, corresponding to a “burst” of beneficial agent being released from the implant. The burst often results in a substantial portion of the beneficial agent, if not all, being released in a very short time, e.g., hours or 1-2 days. Such an initial burst effect can be unacceptable, particularly in those circumstances where sustained delivery is desired, i.e., delivery of beneficial agent over a period of a week or a month or more, or where there is a narrow therapeutic window and release of excess beneficial agent can result in adverse consequences to the subject being treated, or where it is necessary to mimic the naturally-occurring daily profile of beneficial agents, such as hormones and the like, in the body of the subject being treated.

In an attempt to control burst and modulate various stabilizing or release modulating agents, such as metal salts as described in U.S. Pat. Nos. 5,656,297, 5,654,010, 4,985,404 and 4,853,218 have been used. U.S. Pat. No. 3,923,939 describes a method of reducing initial burst of an active agent from a delivery device by removing, prior to implantation, active agent from the exterior surface of the delivery device and through a layer of at least 5% of the overall body thickness extending from the exterior surface of the device.

Notwithstanding some success, those methods have not been entirely satisfactory for the large number of beneficial agents that would be effectively delivered by implants, since in many instances the modulation and stabilization effect is the result of the formation of a complex of the metal ion with the beneficial agent. When such complexes do not form, the stabilization/modulation effect may not be adequate to prevent undesirable “burst” of the beneficial agent upon its introduction into the implant site.

The rapid water uptake into the polymer implant and solvent dispersion into body fluids exhibited by prior devices often results in implants having pore structures that are non-homogeneous in size and shape. Typically, the surface pores take on a finger-like pore structure extending for as much as one-third of a millimeter or more from the implant surface into the implant, and such finger-like pores are open at the surface of the implant to the environment of use. The internal pores tend to be smaller and less accessible to the fluids present in the environment of use. Accordingly, when such devices are implanted, the finger-like pores allow very rapid uptake of aqueous body fluids into the interior of the implant with consequent immediate and rapid dissolution of significant quantities of beneficial agent and unimpeded diffusion of beneficial agent into the environment of use, producing the burst effect discussed above.

Furthermore, rapid water uptake can result in premature polymer precipitation such that a hardened implant or one with a hardened skin is produced. The inner pores and much of the interior of the polymer containing beneficial agent are shut off from contact with the body fluids and a significant reduction in the release of beneficial agent can result over a not insignificant period of time (“lag time”). That lag time is undesirable from the standpoint of presenting a controlled, sustained release of beneficial agent to the subject being treated. What one observes, then, is a burst of beneficial agent being released in a short time period immediately after implantation, a lag time in which no or very little beneficial agent is being released, and subsequently continued delivery of beneficial agent (assuming beneficial agent remains after the burst) until the supply of beneficial agent is exhausted.

With solvent-based depot compositions containing a polymer dissolved in a solvent, the composition solidifies after injection as solvent diffuses from the depot. Since these compositions need to be non-viscous in order to be injected, a large percentage of drug is released as the system forms by diffusion of the solvent, as a “burst” effect.

An additional problem encountered with prior solvent-based depot compositions is that the viscosity of the injectable composition is relatively high, particularly when higher molecular weight polymers are used, and the injection force needed to introduce the composition into a patient's body is therefore high as well (see, e.g. U.S. Pat. No. 6,130,200). To address this problem, those working in the field have employed lower molecular weight polymers and relatively volatile, water-soluble solvents such as ethanol. See, for example, U.S. Pat. Nos. 5,733,950, 5,780,044, and 5,990,194 to Dunn et al and PCT publication WO 98/27962. However, these approaches can result in drug particle settling and/or a higher initial release burst and/or relatively large amounts of emulsifying agent, e.g., about one-third of the total weight of the composition. Furthermore, solvent volatility is problematic from a manufacturing standpoint, and monohydric lower alkanols such as ethanol can denature proteins and peptide drugs. Additionally, the requirement that the bioerodible polymer have a low molecular weight is quite restrictive from a manufacturing standpoint.

Thus, there is still a need for depot composition that allows for reduced initial burst and controlled release of the beneficial agent over a desired period of time.

### SUMMARY

The present invention is directed to the aforementioned needs, and provides compositions and methods for delivering a beneficial agent to a subject by implanting in the subject an implantable system that contains particulates (particles that can be or can include microparticles) having beneficial agent and a burst reducing agent.

In an aspect, a composition for sustained beneficial agent delivery in an animal is provided. The composition includes bioerodible particles and an injectable depot vehicle containing a bioerodible polymer in an organic solvent, for forming a bioerodible depot implant after injection. The bioerodible particles are dispersed in the depot vehicle and contain a beneficial agent and a non-complex release rate controlling agent retarding the release of the beneficial agent from the bioerodible particles. As used herein, a “non-complex” agent means the agent does not contain a complex ion involving a metallic ion having charge interaction with ligands.

In another aspect, a method of forming a composition for sustained beneficial agent delivery in an animal is provided. The method includes preparing an injectable depot vehicle containing a bioerodible polymer in an organic solvent, preparing bioerodible particles comprising a beneficial agent and a non-complex release rate controlling agent, and dispersing the bioerodible particles in the injectable depot vehicle, for forming a bioerodible depot implant after the composition is delivered to an individual, i.e., a patient, such as a person, a domestic animal or wild animal. The bioerodible particles containing a beneficial agent and a non-complex release rate controlling agent retarding the release of the beneficial agent from the bioerodible particles.

In yet another aspect, the present invention provides a method of administering a beneficial agent to an individual in need thereof. The method includes providing a composition that includes bioerodible particles dispersed in an injectable depot vehicle, and injecting into the individual the composition. The depot vehicle contains a bioerodible polymer in an organic solvent. The bioerodible particles contain a beneficial agent and a non-complex release rate controlling agent that retards the release of the beneficial agent from the bioerodible particles.

In another aspect, the present invention provides a composition for sustained beneficial agent delivery in an individual. The composition includes bioerodible particles and an injectable depot vehicle containing a bioerodible polymer in an organic solvent comprising benzyl alcohol, for forming a bioerodible depot implant after injection. The bioerodible particles are dispersed in the depot vehicle and contain a beneficial agent and a non-complex release rate controlling agent retarding the release of the beneficial agent from the bioerodible particles.

In yet another aspect, the present invention provides a composition for sustained beneficial agent delivery in an individual. The composition includes bioerodible particles and an injectable depot vehicle containing a bioerodible polymer in an organic solvent comprising benzyl alcohol, for forming a bioerodible depot implant after injection. The bioerodible particles are dispersed in the depot vehicle and contain subparticles having a beneficial agent and a release rate controlling agent retarding the release of the beneficial agent from the bioerodible particles.

In yet another aspect, a depot composition of the present invention includes bioerodible particles dispersed in the depot vehicle wherein the bioerodible particles contain subparticles compacted together, the subparticles having a beneficial agent and a release rate controlling agent retarding the release of the beneficial agent from the bioerodible particles.

Further, a kit can be provided that includes a depot composition, a syringe and needle for injecting the depot composition into an individual. The composition includes bioerodible particles dispersed in an injectable depot vehicle. The depot vehicle contains a bioerodible polymer in an organic solvent. The bioerodible particles contain a beneficial agent and a release rate controlling agent that retards the release of the beneficial agent from the bioerodible particles.

The release rate controlling agent can hinder movement of the beneficial agent in the bioerodible particles and can hinder access of water to the beneficial agent in the bioerodible particles. In certain embodiments, the release rate controlling agent(s) can function in hindering water access by having property in hydrophobicity, being polymeric to form a gel in physiological condition, thermally reversing between ambient condition and physiological condition, having ionic crosslink network which breaks down by ion exchange, or a combination thereof.

Both the property of the depot vehicle and the property of the particulate in which the beneficial agent is contained contribute to controlling the initial burst and the release rate of the beneficial agent from the depot. First, the depot vehicle, having an organic solvent in the vehicle gel, hinders water from the body fluid to penetrate the depot composition to access the particles. As aqueous fluid slowly replaces the organic solvent, more water is allowed to access the bioerodible particles. Thus, the depot vehicle provides a level of control of release of the beneficial agent, to reduce burst and to control the release rate. The bioerodible particles, having release rate controlling agent, hinders water from accessing the beneficial agent and hinders the release of beneficial agent from the bioerodible particles when aqueous body fluid penetrates the depot composition to reach the bioerodible particles. Thus, the bioerodible particles provide another level of control of initial burst and release rate of the beneficial agent. With release control contribution by both the property of the depot vehicle and the property of the bioerodible particles, the composition of the depot vehicle and the composition of the bioerodible particles can be selected to achieve the burst and release profile desired.

The release rate controlling agents in the above embodiments are preferably a non-complex agent.

### BRIEF DESCRIPTION OF THE DRAWINGS

The foregoing and other objects, features and advantages of the present invention will be more readily understood upon reading the following detailed description in conjunction with the drawings in which objects are not drawn to scale unless specified.

FIG. 1 is a graph illustrating the rheological property (viscosity) of depot vehicle formulations of the present invention (formulations 5-7).

FIG. 2 is a graph illustrating the in vitro dissolution rate of BSA from the alginate formulations of the present invention (formulations 12-15).

FIG. 3 is a graph illustrating the in vitro dissolution rate of hGH with or without complex with Zn ion of the present invention.

FIG. 4 is a graph illustrating the in vitro dissolution rate of hGH/Zn complex compacted with or without stearic acid of the present invention.

FIG. 5 is a graph illustrating the in vivo release profile of bupivacaine obtained from depot formulations of the present invention (formulations 20, 21).

FIG. 6 is a graph illustrating the in vivo release profile of bupivacaine obtained from depot formulations of the present invention (formulations 21, 22).

FIG. 7 is a graph illustrating the in vivo release profile of hGH obtained from depot formulations of the present invention (formulations 23, 24).

FIG. 8 is a graph illustrating the in vivo release profile of hGH obtained from depot formulations of the present invention (formulations 25, 26).

FIG. 9 is a graph illustrating the in vivo release profile of hGH obtained from depot formulations of the present invention (formulations 27, 28).

FIG. 10 is a graph illustrating the in vivo release profile of hGH obtained from depot formulations of the present invention (formulation 29 and 30)

FIG. 11 is a schematic drawing illustrating a depot composition of the present invention.

FIG. 12 is a schematic drawing illustrating another depot composition of the present invention.

FIG. 13 is a schematic drawing illustrating a bioerodible particle of the present invention.

### DETAILED DESCRIPTION

The present invention provides compositions and methods for delivering a beneficial agent to a subject by implanting in the subject an implantable system that contains particulates (particles and/or microparticles) having beneficial agent and a burst reducing agent. The beneficial agent can be delivered systemically or locally to a subject by implanting in the subject an implantable system, which can be formed as a viscous gel or depot particles from a biocompatible polymer and a biocompatible solvent. The beneficial agent is incorporated with the burst reducing agent (or release rate reducing agent) to form particulates, which can be included in the viscous gel or the depot particles. As body fluid gradually penetrates the implant providing an aqueous environment to the beneficial agent in the particulates, the beneficial agent is released to the subject over a prolonged period of time. The composition provides dual control (i.e., by the depot gel and the particles) on the initial burst and the release rate, thus allowing delivery of the beneficial agent with a controlled burst of beneficial agent and sustained release thereafter.

Definitions:

In describing and claiming the present invention, the following terminology will be used in accordance with the definitions set out below.

The singular forms “a,” “an” and “the” include plural referents unless the context clearly dictates otherwise. Thus, for example, reference to “a solvent” includes a single solvent as well as a mixture of two or more different solvents, reference to “a beneficial agent” includes a single beneficial agent as well as two or more different beneficial agents in combination, reference to “an aromatic alcohol” includes a single aromatic alcohol as well as a mixture of two or more different aromatic alcohols, and the like.

The term “beneficial agent” means an agent that effects a desired beneficial, often pharmacological, effect upon administration to a human or an animal, whether alone or in combination with other pharmaceutical excipients or inert ingredients.

As used herein, the term “polynucleotide” refers to a polymeric form of nucleotides of any length, either ribonucleotides or deoxyribonucleotides, and includes double- and single-stranded DNA and RNA. It also includes known types of modifications, substitutions, and internucleotide modifications, which are known in the art.

As used herein, the term “recombinant polynucleotide” refers to a polynucleotide of genomic, cDNA, semisynthetic, or synthetic origin which, by virtue of its origin or manipulation: is not associated with all or a portion of a polynucleotide with which it is associated in nature; is linked to a polynucleotide other than that to which it is linked in nature; or does not occur in nature.

As used herein, the term “polypeptide” refers to a polymer of amino acids, including for example, peptides, oligopeptides, and proteins and derivatives, analogs and fragments thereof, as well as other modifications known in the art, both naturally occurring and non-naturally occurring.

As used herein, the term “purified” and “isolated” when referring to a polypeptide or nucleotide sequence means that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type present.

The term “AUC” means the area under the curve obtained from an in vivo assay in a subject by plotting blood plasma concentration of the beneficial agent in the subject against time, as measured from the time of implantation of the composition, to a time “t” after implantation. The time t will correspond to the delivery period of beneficial agent to a subject.

The term “burst index” means, with respect to a particular composition intended for systemic delivery of a beneficial agent, the quotient formed by dividing (i) the AUC calculated for the first time period after implantation of the composition into a subject divided by the number of hours in the first time period (t₁), by (ii) the AUC calculated for the time period of delivery of beneficial agent, divided by the number of hours in the total duration of the delivery period (t₂). For example the burst index at 24 hours is the quotient formed by dividing (i) the AUC calculated for the first twenty-four hours after implantation of the composition into a subject divided by the number 24, by (ii) the AUC calculated for the time period of delivery of beneficial agent, divided by the number of hours in the total duration of the delivery period.

The phrase “dissolved or dispersed” is intended to encompass all means of establishing a presence of beneficial agent in the gel composition and includes dissolution, dispersion, suspension and the like.

The term “systemic” means, with respect to delivery or administration of a beneficial agent to a subject, that the beneficial agent is detectable at a biologically significant level in the blood plasma of the subject.

The term “local” means, with respect to delivery or administration of a beneficial agent to a subject, that the beneficial agent is delivered to a localized site in the subject but is not detectable at a biologically significant level in the blood plasma of the subject.

The term “gel vehicle” means the composition formed by mixture of the polymer and solvent in the absence of the beneficial agent.

The term “prolonged period” means a period of time over which release of a beneficial agent from the implant of the invention occurs, which will generally be about one week or longer, and preferably about 30 days or longer.

The term “initial burst” means, with respect to a particular composition of this invention, the quotient obtained by dividing (i) the amount by weight of beneficial agent released from the composition in a predetermined initial period of time after implantation, by (ii) the total amount of beneficial agent that is to be delivered from an implanted composition. It is understood that the initial burst may vary depending on the shape and surface area of the implant. Accordingly, the percentages and burst indices associated with initial burst described herein are intended to apply to compositions tested in a form resulting from dispensing of the composition from a standard syringe.

The term “solubility modulator” means, with respect to the beneficial agent, an agent that will alter the solubility of the beneficial agent, with reference to polymer solvent or water, from the solubility of beneficial agent in the absence of the modulator. The modulator may enhance or retard the solubility of the beneficial agent in the solvent or water. However, in the case of beneficial agents that are highly water soluble, the solubility modulator will generally be an agent that will retard the solubility of the beneficial agent in water. The effects of solubility modulators of the beneficial agent may result from interaction of the solubility modulator with the solvent, or with the beneficial agent itself, such as by the formation of complexes, or with both. For the purposes hereof, when the solubility modulator is “associated” with the beneficial agent, all such interactions or formations as may occur are intended. Beneficial agent release rate controlling substances can be such solubility modulators. Solubility modulators may be mixed with the beneficial agent prior to its combination with the viscous gel or may be added to the viscous gel prior to the addition of the beneficial agent, as appropriate.

The terms “subject” and “patient” mean, with respect to the administration of a composition of the invention, an animal or a human being.

Since all solvents, at least on a molecular level, will be soluble in water (i.e., miscible with water) to some very limited extent, the term “immiscible” as used herein means that 7% or less by weight, preferably 5% or less, of the solvent is soluble in or miscible with water. For the purposes of this disclosure, solubility values of solvent in water are considered to be determined at 25° C. Since it is generally recognized that solubility values as reported may not always be conducted at the same conditions, solubility limits recited herein as percent by weight miscible or soluble with water as part of a range or upper limit may not be absolute. For example, if the upper limit on solvent solubility in water is recited herein as “7% by weight,” and no further limitations on the solvent are provided, the solvent “triacetin,” which has a reported solubility in water of 7.17 grams in 100 ml of water, is considered to be included within the limit of 7%. A solubility limit in water of less than 7% by weight as used herein does not include the solvent triacetin or solvents having solubilities in water equal to or greater than triacetin.

The term “bioerodible” or “biodegradable” refers to a material that gradually decomposes, dissolves, hydrolyzes and/or erodes in situ. Generally, the “bioerodible” polymers herein are polymers that are hydrolyzable, and bioerode in situ primarily through hydrolysis.

The term “thixotropic” is used in its conventional sense to refer to a gel composition that can liquefy or at least exhibit a decrease in apparent viscosity upon application of mechanical force such as shear force. The extent of the reduction is in part a function of the shear rate of the gel when subjected to the shearing force. When the shearing force is removed, the viscosity of the thixotropic gel returns to a viscosity at or near that which it displayed prior to being subjected to the shearing force. Accordingly, a thixotropic gel may be subjected to a shearing force when injected from a syringe, which temporarily reduces its viscosity during the injection process. When the injection process is completed, the shearing force is removed and the gel returns very near to its previous state.

A “thixotropic agent” as used herein is one that increases the thixotropy of the composition in which it is contained, promoting shear thinning and enabling use of reduced injection force.

The polymer, solvent and other agents of the invention must be “biocompatible”; that is they must not cause irritation, inflammation or necrosis in the environment of use. The environment of use is a fluid environment and may comprise a subcutaneous, intramuscular, intravascular (high/low flow), intramyocardial, adventitial, intratumoral, or intracerebral portion, wound sites, tight joint spaces or body cavity of a human or animal.

The following definitions apply to the molecular structures described herein:

As used herein, the phrase “having the formula” or “having the structure” is not intended to be limiting and is used in the same way that the term “comprising” is commonly used.

The term “alkyl” as used herein refers to a saturated hydrocarbon group typically although not necessarily containing 1 to about 30 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of 1 to 6 carbon atoms, preferably 1 to 4 carbon atoms. “Substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl or lower alkyl.

The term “aryl” as used herein, and unless otherwise specified, refers to an aromatic substituent containing a single aromatic ring or multiple aromatic rings that are fused together, linked covalently, or linked to a common group such as a methylene or ethylene moiety. Preferred aryl groups contain one aromatic ring or two fused or linked aromatic rings, e.g., phenyl, naphthyl, biphenyl, diphenylether, diphenylamine, benzophenone, and the like, and most preferred aryl groups are monocyclic. “Substituted aryl” refers to an aryl moiety substituted with one or more substituent groups, and the terms “heteroatom-containing aryl” and “heteroaryl” refer to aryl in which at least one carbon atom is replaced with a heteroatom. Unless otherwise indicated, the term “aryl” includes heteroaryl, substituted aryl, and substituted heteroaryl groups.

The term “aralkyl” refers to an alkyl group substituted with an aryl group, wherein alkyl and aryl are as defined above. The term “heteroaralkyl” refers to an alkyl group substituted with a heteroaryl group. Unless otherwise indicated, the term “aralkyl” includes heteroaralkyl and substituted aralkyl groups as well as unsubstituted aralkyl groups. Generally, the term “aralkyl” herein refers to an aryl-substituted lower alkyl group, preferably a phenyl substituted lower alkyl group such as benzyl, phenethyl, 1-phenylpropyl, 2-phenylpropyl, and the like.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon. Similarly, the term “heterocyclic” refers to a cyclic substituent that is heteroatom-containing, the term “heteroaryl” refers to an aryl substituent that is heteroatom-containing, and the like.

By “substituted” as in “substituted alkyl,” “substituted aryl” and the like, as alluded to in some of the aforementioned definitions, is meant that in the alkyl or aryl moiety, respectively, at least one hydrogen atom bound to a carbon atom is replaced with one or more non-interfering substituents such as hydroxyl, alkoxy, thio, amino, halo, and the like.

Injectable Depot Compositions

In contrast to prior polymer-based injectable depots, depots of the present invention have beneficial agent containing particulates that modulate a release rate. Injectable depot compositions for delivery of beneficial agents over a prolonged period of time may be formed as viscous compositions that can be gel like prior to injection of the depot into a subject. The viscous gel for an implant supports dispersed particulates having beneficial agent to provide appropriate delivery profiles. The depot compositions include those having low initial burst of the beneficial agent as the beneficial agent is released from the depot over time.

Various factors can be adjusted to achieve the low initial burst of beneficial agent release. First, the initial burst can be controlled by factors related to the property of the depot vehicle, such as the water immiscibility of the solvent, polymer/solvent ratio, and the property of the polymer. The extent of water immiscibility of the solvent used in the depot vehicle affects that rate aqueous body fluid can penetrate the depot to release the beneficial agent. Generally, higher water immiscibility leads to a lower initial burst and a slower beneficial agent release rate.

Further, varying the molecular weight of the polymer in the depot vehicle, or adjusting the molecular weight distribution of the polymer material in the depot vehicle can affect the initial burst and the release rate of beneficial agent from the depot. Generally, a higher molecular weight polymer renders a lower initial burst and slower release rate of the beneficial agent.

The inclusion of beneficial agent containing particulates provides additional tools to further control the initial burst and the release rate. When the beneficial agent is confined in particulates that are enclosed (e.g., dispersed) within the depot implant, the property of the particulates will further affect how fast the beneficial agent is allowed to leave the particulates to diffuse through the depot vehicle material. The chemical nature of the material included in the particulates has an impact on the beneficial agent release rate. Factors such as the particle size, the disintegration of the particulates, the morphology of the particulates (e.g., whether pores are present in the particulates before implanting or can be formed easily by body fluid attack), coatings, complex formation by the beneficial agent and the strength of complex bond, can be manipulated to achieve the desired low initial burst and release rate.

Both the property of the depot vehicle and the property of the particulate in which the beneficial agent is contained contribute to controlling the initial burst and the release rate of the beneficial agent from the depot. Typically, the mechanism of control is dominated by the property of the particulates initially. In other words, the rate controlling factor is the property of the particulates initially. In the beginning phase of depot implantation, due to the integrity of the particulates, the beneficial agent leaves the particulates with difficulty. But once the beneficial agent leaves a particle, it tends to diffuse away relatively easily. However, in the later phase of implantation, the particulates have disintegrated to the extent that the beneficial agent is practically not hindered by the particles any longer. The rate controlling factors are then primarily those associated with the diffusion of the agents within the depot vehicle material, akin to a situation in which the beneficial agent is merely present in the depot without the particulate form.

The composition of the depot vehicle and the composition of the bioerodible particles can be selected to achieve the burst and release profile desired. For example, the solvent and the depot polymer can be selected depending on whether a fast release is desired. A solvent that is quickly replaced by aqueous medium when implanted would result in fast release initially. Higher molecular weight polymer in the depot polymer will tend to provide slower initial release rate. Further, a more hydrophobic polymer will tend to retard water movement within the depot implant and lead to a slower release.

The present invention also provides several embodiments of release rate controlling agents that can retard release rate of the beneficial agent from the bioerodible particles, as compared to when such release rate controlling agents are absent. Further, generally, a higher ratio of the release rate controlling agent to the beneficial agent will provide a slower beneficial agent release rate. Thus, the composition of the bioerodible particles can be selected in conjunction with selecting the composition of the depot vehicle to effect the release rate of the beneficial agent, depending on whether a fast release rate or a slower release rate is desired.

Particulates of beneficial agent(s) with burst reducing agents (or release rate controlling agents), such as excipient, polymer, binding agents, etc., can be made by adapting conventional methods of making particles including a beneficial agent. Generally, the particulates are biodegradable or bioerodible that after a long implantation period the particulates will disintegrate such that all the particulates are absorbed or metabolized by the body and no insoluble material remains. Although possible, it is not desirable that any nonabsorbable nonmetabilizable material be included in the particulates.

An example of forming particulates having burst reducing agent or release rate-controlling agent (e.g., polymer) and beneficial agent is by removal of solvent from a solution of dissolved burst reducing agent and beneficial agent. After the removal of the solvent, solid particulates are formed. Such particulates are typically dry. Although heat can be used to facilitate the removal of solvent, to prevent or reduce degradation of the beneficial agent in the drying (solvent removal) process, preferably excess heat is not used. Thus, preferably methods of solvent removal are by spray-drying or freeze-drying. Further, drying by negative pressure (i.e., applying a vacuuming force) to remove the solvent can also be used, regardless of whether the solution has been frozen (as in lyophilization). Vacuuming, spray-drying and lyophilization methods are known in the art and can be applied to form the particulates of a burst reducing agent and beneficial agent in view of the teachings of the present invention. Other methods of making particulates forming droplets of the burst reducing agent/beneficial agent in a solvent and freezing the droplets in a non-solvent, and then extracting the solvent, as describe, e.g., in U.S. Pat. No. 5,019,400.

Generally, the burst reducing agent (or burst controlling agent) is less soluble in body fluid under physiological condition than the beneficial agent or would render the beneficial agent less soluble when associated with the burst reducing agent (for example, by bond formation). One embodiment of a particulate having beneficial agent that can be incorporated into the depot of the present invention is the kind that has beneficial agent (e.g., proteins such as hormones) chelated with multi-valence metal ions to form stable complexes, such as hGH/Zn. Suitable multi-valence ions include divalent metal ions such as zinc, magnesium, calcium, copper, iron, aluminum and the like. Suitable multi-valent substances that can be used include magnesium hydroxide, magnesium carbonate, calcium carbonate, zinc carbonate, magnesium acetate, zinc acetate, magnesium chloride, zinc chloride, magnesium sulfate, zinc sulfate, magnesium citrate and zinc citrate, copper sulfate, calcium carbonate, calcium hydroxide, calcium chloride, ferric chloride, aluminum oxide and the like. For example, a protein-zinc complex in buffer solution (e.g., hGH solution) can be formed by spray drying or lyophilization using conventional techniques. Suitable buffer solution, e.g., TRIS buffer solutions (5 or 50 mM) suitable for a particular beneficial agent is used, and the multi-valence ions (e.g., zinc ions from zinc acetate) are added to complex with the beneficial agent before particle formation. Typically, the amount of multi-valence agent to beneficial agent is about 0.5/1 to 100/1, preferably about 1/1 to 50/1 by molar ratio.

Other than using zinc ions for complexing, complexing can be beneficially done using other multivalent ions to achieve reduction of initial burst and controlling release rate of the beneficial agent. Other non-zinc or non-complex methodologies can be used to reduce the initial burst and controlling release of the beneficial agent as well. Generally such methodologies achieve the goal by hindering water from ready access to the beneficial agent. Such water hindering can be done by a physical barrier or chemical barrier. Examples of physical barriers are particle structures that will hydrolyze slowly, thus hindering the free movement of water and beneficial agent molecules. An example of a chemical barrier is one in which a hydrophobic environment is provided by an excipient such as a polymer or a relatively insoluble agent.

One non-zinc non-complex embodiment of particulates suitable for the depot implant of the present invention is achieved by protecting the beneficial agent from aqueous environment using a hydrophobic excipient. One way to achieve this end is forming the particulates by compression of the beneficial agent with fatty acid(s). Beneficial agents, such as protein or polypeptide drugs can be compressed with certain hydrophobic excipients such as fatty acids, e.g., stearic acid, or esters of fatty acids, e.g. ethyl stearate. The hydrophobic excipient is more hydrophobic than the beneficial agent and will tend to allow less water access than the beneficial agent, thereby slowing the disintegration of the particulates. The hydrophobic nature of the excipient fatty acid or ester of fatty acid inhibits water access to the particulates, thereby retarding the disintegration of the particles and release of the beneficial agent. Suitable fatty acids are stearic acid, palmitic acid, margaric acid, oleic acid and the like. Suitable esters of fatty acids are ethyl stearate, ethyl palmitate, ethyl margarate, ethyl oleate, methyl oleate, methyl palmitate, methyl stearate and the like, and stearic acid being preferred. Generally, the fatty acid to the beneficial agent molar ratio is about 0.1/1 to 10/1, preferably about 0.5/1 to 5/1. For example, a beneficial agent, human growth hormone (hGH) particles are prepared by lyophilized a solution of hGH. The lyophilized hGH and about equal weight of stearic acid are blended and ground. The ground material is then compressed to form tablets. The compressed tablets are then ground and sieved through screen to achieve the desired size range, e.g., between about 30-250 microns (μm). Alternatively, the fatty acid and the beneficial agent can be dissolved together in solution and dried by either vacuum or heat or combination of both. Lyophilization can also be used for drying. The formed particles can then be compressed, ground and sieved. It is understood that particulates with other beneficial agents can be made in a similar way.

Another non-zinc-complexing methodology is to provide particulates with a network that hinders (or traps) the beneficial agent from free movement. For example, alginate can be used for forming ionic crosslinked network with calcium ions. The beneficial agent can be released through ion exchange and slowly diffuse out of the network. In ion exchange, the calcium ions are replaced with another cation, e.g., sodium ion, thereby slowly breaking the ionic network. In the body, sodium ions from the body gradually replace calcium ions in the calcium alginate. In making the network, a buffer solution of a pH in which a beneficial agent is stable is used to include the beneficial agent and the alginate. Generally, such alginate particulates are formed by mixing a beneficial agent solution with a sodium alginate solution followed by addition of a calcium solution (e.g., calcium lactate). The amount of calcium ions to the sodium alginate, as well to the beneficial agent can be varied to achieve the desired ionic cross-link for the desired physical property. Typically, the beneficial agent to alginate weight ratio is about 5/1 to 1/100, preferably about 1/1 to 1/10. The weight ratio of sodium alginate to calcium lactate is about 1/1 to 1000/1, preferably about 10/1 to 100/1. The pH of the solution is typically about 5-9, preferable about 7. The concentration of alginate solution is typically about 0.5% to 10% by weight, preferably about 1% to 5%. The concentration of beneficial agent is typically about 0.1%-10% by weight, preferably about 0.5% to 5%. The mixture can then be dried, e.g., lyophilized. The resulting lyophilized material then can be sieved for the desired particle size, e.g., 30-250 microns. If desired, the lyophilized particles can be compacted (compressed) into tablets and then ground and sieved to achieve the right particle size, e.g., 30-250 microns; 38-212 microns, etc.

Yet another embodiment of a non-zinc complexing methodology is the application of particulates that contains thermally reversible materials. An example of a thermally reversible material is a poloxamer, e.g., PLURONIC F127 (BASF, USA). Poloxamers are nonionic polyoxyethylene-polyoxypropylene (PEO-PPO) block co-polymers and have reported to be helpful in stabilizing proteins. Triblock PEO-PPO-PEP copolymers have a central hydrophobic PPO segment and hydrophilic PEO segment at the ends. Such polymers in an appropriate aqueous environment have a fluid consistency at room temperature. At a higher temperature, e.g., at human body temperature it turns into a gel. Thus, when particulates of the thermally reversible material is implanted as part of a depot into the body, when body fluid gradually penetrates the depot to access the particulates, the particulates, although gradually absorb water and turn into gel form, remains in particle shape for a long period and affords both stability to the beneficial agent and controls the release thereof from the particulates.

For example, the poloxamer PLURONIC F127 can be used. PLURONIC F127 has been reported to enhance the stability of proteins such as urease and interleukin-2. See, Kikwai, Loice et al., In Vitro and In Vivo Evaluation of Topical Formulations of Spantide II, AAPS PharmSciTech. 2005; 6(4):E565-E572. However, PLURONIC F127 has not been used previously in particles in a depot composition. PLURONIC F127 is an example of thermally reversible material that can be used for making the particulates of the present invention.

Other thermally reversible material that can be used for forming the particulates of the present invention are those reported in Cheng et al. (U.S. Patent Publication 20040029994), which is incorporated by reference in its entirely herein. The thermally reversible material has a core of A and arms of B, or vice verse. A may be a homopolymer or copolymer (either linear or branched) that is soluble over a range of conditions in the environment of interest, e.g., in room temperature and in the physiological condition in the body. A may be made of material selected from e.g., the group of polyethylene glycol (PEG), polyvinyl pyrrolidone, polyvinyl alcohol, polyhydroxyethylmethacrylate, and hyaluronic acid. B is a polymeric material that responds to an environment suited to the intended application of the invention, for example, water-solubility under ambient conditions and aggregation under physiologic conditions. The environmental condition triggers the switch in water solubility/insolubility between ambient and physiological conditions. Such conditions can be factors such as pH, temperature, ionic strength, and combinations thereof. As B switches in solubility, the thermally reversible material also switches in solubility. Examples of B include a material selected from a group consisting of poly-N-isopropyl acrylamide (PNIPAAm), which is a temperature responsive polymer, hydroxypropylmethyl cellulose and other methyl cellulose derivatives, poly(ethylene glycol vinyl ether-co-butyl vinyl ether), polymers of N-alky acrylamide derivatives, poly(amino acid)s or peptide sequences such as silk and elastin peptides, poly(methacryloy L-alanine methyl ester), poly(methacryloy L-alanine ethyl ester).

Generally, the thermally reversible polymer is dissolved in a buffer along with the beneficial agent and the solution is then dried to result in a dry material, which is sieved to the desired particle size (e.g., between about 30-250 microns), either with compression/grinding or without compression/grinding. The drying can be done, e.g., by spray drying or by lyophilization. Typically, the weight ratio of the beneficial agent (e.g., a hormone, such as hGH) to the thermally reversible polymer is about 2/1 to 1/20, preferably about 1/2 to 1/10. The dried particles can then be included in a depot vehicle for implantation. Thus, thermal reversible polymers provide a unique material that can easily be made into a beneficial agent containing solution and then formed into particulates that when implanted into the body will form gel particles, thus permitting sustained release of the beneficial agent.

If desired, a combination of the techniques for reducing initial burst and controlling release rate can be combined. For example, fatty acids can be blended with compressed with particulates formed by the other techniques, such as zinc complexing, using thermally reversible polymers, etc.

In forming the particulates, typically the burst reducing agent and beneficial agent are present in a burst reducing agent to beneficial agent weight ratio of about 1/100 to about 100/1, preferably about 1/10 to about 10/1, more preferably about 1/5 to about 5/1, even more preferably about 1/2 to about 2/1. The burst reducing agent and beneficial agent are present in a solution environment suitable for the beneficial agent and in which the beneficial agent is stable, such as at a concentration in a buffer in the pH range in which the beneficial agent (e.g., protein) is stable. In view of the present disclosure, for any given beneficial agent, the appropriate buffer, pH and concentration ranges can be determined by one skilled in the art without undue experimentation.

Generally the beneficial agent is present in the solution before solvent removal at a concentration of about 0.1 wt % to about 30 wt %, preferably about 0.5 wt % to about 20 wt %, more preferably about 1 wt % to about 10 wt %.

In order to produce particulates that will release the beneficial agent in a more uniform controlled release rate, it is preferred that the solid material formed by solvent removal be processed to obtain particulates of particular particle size ranges. Various ways to separate the particles into different groups of particle size and/or density ranges can be used. For example, sieving with screens with openings of particular sizes can be used. Other processes such as grinding to reduce the particle size and separation by air movement, e.g., air cyclone, can be used.

A further method to control the initial burst and reduce the release rate is to provide beneficial-agent-containing particulates that are larger in size and denser. A larger denser particle will require a longer time for the body fluid to penetrate and the beneficial agent to escape therefrom. Particulates that are made by spray drying and lyophilization tend to have a lot of void space. Compacting (or compressing) fluffy materials obtained by processes such as spray-drying and freeze-drying into denser materials such as tablets and then reducing the size to the desired range, e.g., by grinding and sieving, can provide control of the density of the resultant particulates. Thus, many of the particulates will contain smaller particles compacted together. Typically the particulates and the smaller particles from which the particulates derive contain substantially the small material chemically. The appropriate particle size and density for such compacted material will facilitate dispersion in the depot vehicle and result in the desired beneficial agent release rate.

Typically, the particulates are in the range of about 1 to about 200 microns, preferably about 5 to about 150 microns, more preferably about 30 to about 120 microns.

The particulates are included in the deport formulation composition for implanting, for example, by injection with a syringe and needle. The depot composition is formulated so that the depot composition can be readily implanted (e.g., by injection) into the desired location to form a mass that can remain in place for the period suitable for controlled release of the beneficial agent and for any additional benefit of mechanical support if applicable. The mechanical and rheological properties suitable for injectable depot compositions are known in the art. Typically, the polymer of the depot vehicle with particulates are present in an appropriate amount of solvent such that the depot composition can be so implanted.

Typically, the depot vehicle and particulates are present such that the desired amount of beneficial agent can be held and is gradually released over a desired length of time at a controlled rate of release. In the depot, the particulates that contain the beneficial agent and the burst reducing agent are present at a particulate to gel composition weight percent of about 1% to about 50%, preferably about 5% to about 30%, even more preferably about 10% to about 20%.

Another way to formulate a particulate is to encapsulate the formulated burst reducing agent-beneficial agent (e.g. drug) above with a biodegradable polymer, such as PLGA RG 502, RG 752, etc. (which are known to one skilled in the art), with conventional encapsulation techniques to form depot particles. The beneficial agent can be formed into subparticles first either by itself, with other excipients, or with a burst reducing agent mentioned above. In this way, depot particles can be formed that each contain a plurality of burst reducing agent-beneficial agent particulates. The depot particles can be further incorporated into an injectable depot vehicle that will gel after introduction into the implant site. When such depot particles are incorporated into a depot vehicle, the resulting depot material will provide an additional level of control of release rate because each level of release control (within the burst reducing agent-beneficial agent particulate, within the depot particle, and within the depot vehicle mass) can be adjusted to suit the desired release profile. It is noted that the polymers described for forming the depot vehicle can also be used to form the depot particles, employing the suitable solvent. Different polymers can be used for forming the depot particle and forming the depot gel to provide more control of the release rate. Of course, if desired, the depot particles can also be delivered directly into a site in the animal body.

Particles that contain subparticles can also be formed with the burst reducing agents (or release rate controlling agents) mentioned above. Burst reducing agents can be present in either or both the particles and subparticles. Burst reducing agents can be present within the subparticles and being different from those outside the subparticles.

Briefly, encapsulation can be done by dispersing beneficial agent particulates or burst reducing agent-beneficial agent particulates in a polymer solution (e.g., PLGA solution). The beneficial agent particulates or burst reducing agent-beneficial agent particulates can be dispersed in the polymer solution and the encapsulated particles can be formed by various processes such as spray drying, spray freezing/solvent extraction, oil-in-water emulsion and evaporation etc., The polymer for the encapsulation is selected to be insoluble or sparingly soluble in the solvent for the depot vehicle. In this way, after the depot particles are formed (with the burst reducing agent-beneficial agent particulates encapsulated therein), they will not disintegrate quickly within the depot vehicle. Biodegradable polymers used in the encapsulation of beneficial agent or combination of beneficial/burst reducing agent include polymers which are insoluble or sparsely soluble in the solvent used for making depot vehicles, such as PLGA with L/G ratio less than 50/50, polydioxanone (PDO) and the like. The encapsulated particles with beneficial agent or combination of beneficial/burst reducing agent are then dispersed in a depot vehicle of biodegradable polymer such as PLGA RG 502, in water-insoluble solvent such as benzyl benzoate.

Generally, the depot particles with encapsulated burst reducing agent-beneficial agent particulates includes about 5 wt % to about 80 wt %, preferably about 10 wt % to about 60 wt %, more about 20 wt % to about 50 wt % of the burst-reducing agent-beneficial agent particulates. The depot particles can have particle size of about 1 to about 250 microns, preferably about 10 to about 150 microns, more preferably about 30 to about 125 microns.

Yet another method of forming bioerodible particles to be included in a depot gel implant is to coat particles having beneficial agent(s) with a coating that hinders body fluid from accessing and releasing the beneficial agent. Coating method for coating particles known in the art can be used.

FIG. 11 illustrates a depot composition of the present invention. In FIG. 11, the depot composition 100 includes bioerodible particles 104 dispersed in a gel 108. FIG. 12 illustrates another depot composition 120 having a gel 122 within which is dispersed bioerodible particles 124, which in turn include subparticles 128 in a carrier matrix 132. FIG. 13 illustrates a particle 138 having a coating 142 of a burst reducing agent enclosing a core 144, which contains a beneficial agent. The particle 138 can be formed by coating a core particle (core 144) that contains the beneficial agent. The core may or may not contain another burst reducing agent.

After an injectable depot gel composition is prepared, typically, the viscous gel will be injected from a standard hypodermic syringe that has been pre-filled with the viscous gel composition to form the depot. The gel contains depot vehicle containing particulates (particles or microparticles), which have burst reducing agent and a beneficial agent (or drug). It is often preferred that injections take place using the smallest size needle (i.e., smallest diameter) to reduce discomfort to the subject when the injection takes place through the skin and into subcutaneous tissue. It is desirable to be able to inject gels through needles ranging from 16 gauge and higher, preferably 20 gauge and higher, more preferably 22 gauge and higher, even more preferably 24 gauge and higher. With highly viscous gels, i.e., gels having a viscosity of about 200 poise or greater, injection forces to dispense the gel from a syringe having a needle in the 20-30 gauge range may be so high as to make the injection difficult or reasonably impossible when done manually. At the same time, the high viscosity of the gel is desirable to maintain the integrity of the depot after injection and during the dispensing period and also facilitate desired suspension characteristics of the beneficial agent in the gel.

The Bioerodible, Biocompatible Polymer:

Polymers that are useful in conjunction with the methods and compositions of the invention are bioerodible, i.e., they gradually hydrolyze, dissolve, physically erode, or otherwise disintegrate in the presence of the aqueous fluids of a patient's body. Generally, the polymers bioerode as a result of hydrolysis or physical erosion, although the primary bioerosion process is typically hydrolysis.

Such polymers include, but are not limited to, polylactides, polyglycolides, polycaprolactones, polyanhydrides, polyamines, polyurethanes, polyesteramides, polyorthoesters, polydioxanones, polyacetals, polyketals, polycarbonates, polyphosphoesters, polyorthocarbonates, polyphosphazenes, succinates, poly(malic acid), poly(amino acids), polyvinylpyrrolidone, polyethylene glycol, polyhydroxycellulose, chitin, chitosan, hyaluronic acid, and copolymers, terpolymers and mixtures thereof.

Presently preferred polymers are polylactides, that is, a lactic acid-based polymer that can be based solely on lactic acid or can be a copolymer based on lactic acid glycolic acid and/or caprolactone, and which may include small amounts of other comonomers that do not substantially affect the advantageous results that can be achieved in accordance with the present invention. As used herein, the term “lactic acid” includes the isomers L-lactic acid, D-lactic acid, DL-lactic acid and lactide, while the term “glycolic acid” includes glycolide. Most preferred are poly(lactide-co-glycolide)copolymers, commonly referred to as “PLGA.” The polymer may have a monomer ratio of lactic acid/glycolic acid (L/G) of from about 100:0 to about 15:85, preferably from about 75:25 to about 30:70, more preferably from about 60:40 to about 40:60, and an especially useful copolymer has a monomer ratio of lactic acid/glycolic acid of about 50:50.

The poly(caprolactone-co-lactic acid) (PCL-co-LA) polymer has a comonomer ratio of caprolactone/lactic acid of from about 10:90 to about 90:10, from about 50:50; preferably from about 35:65 to about 65:35; and more preferably from about 25:75 to about 75:25. In certain embodiments, the lactic acid based polymer comprises a blend of about 0-90% caprolactone, about 0-100% lactic acid, and about 0-60% glycolic acid.

The lactic acid-based polymer has a number average molecular weight of from about 1,000 to about 120,000, preferably from about 5,000 to about 50,000, more preferably from about 8,000 to about 30,000, as determined by gel permeation chromatography (GPC). In contrast to prior polymer-based injectable depots, the present invention allows use of higher molecular weight polymers, insofar as the aromatic alcohol of the composition provides excellent shear thinning even with high molecular weight polymers. As indicated in aforementioned U.S. Pat. No. 5,242,910, the polymer can be prepared in accordance with the teachings of U.S. Pat. No. 4,443,340. Alternatively, the lactic acid-based polymer can be prepared directly from lactic acid or a mixture of lactic acid and glycolic acid (with or without a further comonomer) in accordance with the techniques set forth in U.S. Pat. No. 5,310,865. The contents of all of these patents are incorporated by reference. Suitable lactic acid-based polymers are available commercially. For instance, 50:50 lactic acid:glycolic acid copolymers having molecular weights of 8,000, 10,000, 30,000 and 100,000 are available from Boehringer Ingelheim (Petersburg, Va.), Medisorb Technologies International L.P. (Cincinatti, Ohio) and Birmingham Polymers, Inc. (Birmingham, Ala.) as described below.

Examples of useful polymers include, but are not limited to, Poly (D,L-lactide) Resomer® L104, PLA-L104, Poly (D,L-lactide-co-glycolide) 50:50 Resomer® RG502, Poly (D,L-lactide-co-glycolide) 50:50 Resomer® RG502H, PLGA-502H, Poly (D,L-lactide-co-glycolide) 50:50 Resomer® RG503, PLGA-503, Poly (D,L-lactide-co-glycolide) 50:50 Resomer® RG506, PLGA-506, Poly L-Lactide MW 2,000 (Resomer® L 206, Resomer® L 207, Resomer® L 209, Resomer® L 214); Poly D,L Lactide (Resomer® R 104, Resomer® R 202, Resomer® R 203, Resomer® R 206, Resomer® R 207, Resomer® R 208); Poly L-Lactide-co-D,L-lactide 90:10 (Resomer® LR 209); Poly glycolide (Resomer® G 205); Poly D,L-lactide-co-glycolide 50:50 (Resomer® RG 504H, Resomer® RG 504, Resomer® RG 505); Poly D-L-lactide-co-glycolide 75:25 (Resomer® RG 752, PLGA-755, Resomer® RG 756); Poly D,L-lactide-co-glycolide 85:15 (Resomer® RG 858); Poly L-lactide-co-trimethylene carbonate 70:30 (Resomer® LT 706); Poly dioxanone (Resomer® X 210) (Boehringer Ingelheim Chemicals, Inc., Petersburg, Va.).

Additional examples include, but are not limited to, DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L.P., Cincinatti, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ε-caprolactone; Poly DL-lactide-co-caprolactone 25:75; and Poly DL-lactide-co-caprolactone 75:25 (Birmingham Polymers, Inc., Birmingham, Ala.).

The biocompatible polymer is present in the gel vehicle composition in an amount ranging from about 5 to about 90% by weight, preferably from about 10 to about 85% by weight, preferably from about 15 to about 80% by weight, preferably from about 20 to about 75% by weight, preferably from about 30 to about 70% by weight and typically from about 35 to about 65% by weight of the viscous gel, the viscous gel comprising the combined amounts of the biocompatible polymer and the aromatic alcohol. The solvent will be added to polymer in amounts described below, to provide implantable or viscous gels. The aromatic alcohol enables a much wider range of polymer/solvent ratios than obtainable previously.

Solvents and Thixotropic Agents:

The injectable depot compositions of the invention can contain a water-immiscible solvent having a miscibility in water that is less than 7 wt % at 25° C., in addition to the bioerodible polymer, and other ingredients such as excipients and other polymers. The solvent must be biocompatible, should form a gel, preferably a viscous gel with the polymer, and restrict water uptake into the implant. Suitable solvents will substantially restrict the uptake of water by the implant and, as noted above, may be characterized as immiscible in water, i.e., having a solubility or miscibility in water of at most 7% by weight. Preferably, the water solubility of the aromatic alcohol is 5 wt % or less, more preferably 3 wt % or less, and even more preferably 1 wt % or less. Most preferably, the solubility of the aromatic alcohol in water is equal to or less than 0.5 weight percent. In preferred embodiments, the solvent is selected from the group consisting of an aromatic alcohol, esters of aromatic acids, aromatic ketones, and mixtures thereof.

Water miscibility may be determined experimentally as follows: Water (1-5 g) is placed in a tared clear container at a controlled temperature, about 25° C., and weighed, and a candidate solvent is added dropwise. The solution is swirled to observe phase separation. When the saturation point appears to be reached, as determined by observation of phase separation, the solution is allowed to stand overnight and is re-checked the following day. If the solution is still saturated, as determined by observation of phase separation, then the percent (w/w) of solvent added is determined. Otherwise more solvent is added and the process repeated. Solubility or miscibility is determined by dividing the total weight of solvent added by the final weight of the solvent/water mixture. When solvent mixtures are used, they are pre-mixed prior to adding to the water.

A suitable solvent can be an aromatic alcohol having the structural formula (I)
Ar-(L)ₙ-OH (I)

wherein Ar is a substituted or unsubstituted aryl or heteroaryl group, n is zero or 1, and L is a linking moiety. Preferably, Ar is a monocyclic aryl or heteroaryl group, optionally substituted with one or more noninterfering substituents such as hydroxyl, alkoxy, thio, amino, halo, and the like. More preferably, Ar is an unsubstituted 5- or 6-membered aryl or heteroaryl group such as phenyl, cyclopentadienyl, pyridinyl, pyrimidinyl, pyrazinyl, pyrrolyl, pyrazolyl, imidazolyl, furanyl, thiophenyl, thiazolyl, isothiazolyl, or the like. The subscript “n” is zero or 1, meaning that the linking moiety L may or may not be present. Preferably, n is 1 and L is generally a lower alkylene linkage such as methylene or ethylene, wherein the linkage may include heteroatoms such as O, N or S. Most preferably, Ar is phenyl, n is 1, and L is methylene, such that the aromatic alcohol is benzyl alcohol.

In another embodiment, the injectable depot composition of the invention contains, in addition to the biocompatible, bioerodible polymer and the beneficial agent, (1) a solvent selected from the group consisting of esters of aromatic acids, aromatic ketones, and mixtures thereof, which has miscibility in water of less than or equal to 7% at 25° C., and is present in an amount effective to plasticize the polymer and form a gel therewith, and (2) an effective thixotropic amount of an aromatic alcohol as described above. Generally, the weight ratio of the aromatic alcohol to the ester or ketone is in the range of about 1% to about 99%, preferably in the range of about 10% to about 90%, preferably in the range of about 20% to about 80%, preferably in the range of about 25% to about 75%, often in the range of about 25% to about 50%. In this case, the aromatic alcohol serves primarily as a thixotropic agent, but also acts as a co-solvent for the bioerodible polymer. The injectable composition of the first embodiment, this composition is preferably free of monohydric lower alkanol. Such lower alkanol solvents are volatile, tending to cause problems during manufacture, and are potentially denaturing to or otherwise reactive with the beneficial agent. Preferably the compositions are also free of solvents having miscibility in water that is greater than 7 wt. % at 25° C.

Preferred solvents include derivatives of benzoic acid and include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate. The more preferred solvents are benzyl benzoate (“BB”), benzyl alcohol (“BA”), ethyl benzoate (“EB”), mixtures of BB and BA, mixtures of BB and ethanol, and mixtures of BB and EB.

We have observed that when benzyl alcohol was used as the solvent, in contrast to formulations using benzyl benzoate and benzyl benzoate with ethanol as a thixotropic agent, respectively, significantly reduced injection force was needed for injection. Notably, due to the shear thinning behavior, the formulations using benzyl alcohol as the solvent, versus benzyl benzoate with ethanol, as a thixotropic agent showed significantly reduced injection force while maintaining viscosities equal to or greater than the formulations using benzyl benzoate, at lower shear rate; thus maintaining the intactness of the depot after injection into the animals. Also, when benzyl alcohol was mixed with benzyl benzoate in various proportions in the solvent, for any given polymer, the injection force decreases as the percentage of the benzyl alcohol increased.

Particle size and amount of loading of the beneficial agent, i.e. drug, are additional factors affecting the injection force of the depot formulation. We have found that that the injection force of the depot formulations increases with the increase of drug particle loading. For example, using depot gel lysozyme containing differing amounts (5-30% loading) and particle sizes (5-50 microns) of lysozyme and testing for injection force using 27 gauge, 2″ needles, injecting at 1 milliliters (ml) per minute, we found that with 10 wt % particle loading, the injection forces increased about 50% compared to the corresponding gel formulation, regardless of the composition of the gel formulation. For any given particle loading, the injection force was dependent on the proportion of benzyl alcohol in the gel formulation. This indicates that benzyl alcohol significantly reduces the injection force of the depot gel formulations of the invention. Thus, benzyl alcohol, can be beneficially used for injectable depot compositions having particles to allow easier injection of the depot composition due to reduced injection force.

The composition may also include, in addition to the water-immiscible solvent(s), one or more additional miscible solvents (“component solvents”), provided that any such additional solvent is other than a lower alkanol. Component solvents compatible and miscible with the primary solvent(s) may have a higher miscibility with water and the resulting mixtures may still exhibit significant restriction of water uptake into the implant. Such mixtures will be referred to as “component solvent mixtures.” Useful component solvent mixtures may exhibit solubilities in water greater than the primary solvents themselves, typically between 0.1 weight percent (wt %) and up to and including 50 weight percent, preferably up to and including 30 weight percent, and most preferably up to an including 10 weight percent, without detrimentally affecting the restriction of water uptake exhibited by the implants of the invention.

Component solvents useful in component solvent mixtures are those solvents that are miscible with the primary solvent or solvent mixture, and include, but are not limited, to triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glycerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, glycofurol, dimethyl sulfoxide, tetrahydrofuran, caprolactam, decylmethylsulfoxide, oleic acid, and 1-dodecylazacyclo-heptan-2-one, and mixtures thereof.

In an especially preferred embodiment, the solvent is selected from lower alkyl and aralkyl esters of benzoic acid, the aromatic alcohol is present, serving as a thixotropic agent, and the polymer is a lactic-acid based polymer, most preferably PLGA, having a number average molecular weight of between about 1,000 to about 120,000, preferably about 5,000 to 50,000, more preferably about 8,000 to 30,000. Presently, the most preferred solvents are benzyl benzoate and the lower alkyl esters of benzoic acid, the most preferred thixotropic agent is benzyl alcohol, as noted earlier herein.

The solvent or solvent mixture is capable of dissolving the polymer to form a viscous gel that can maintain particles with the beneficial agent dissolved or dispersed and isolated from the environment of use prior to release. The compositions of the present invention provide implants having a low burst index. The use of a solvent or component solvent mixture that solubilizes or plasticizes the polymer but substantially restricts uptake of water into implant is a factor that reduces burst in the release of the beneficial agent.

In the material for the depot vehicle, the solvent or solvent mixture is typically present in an amount of from about 95 to about 5% by weight, preferably about 75 to about 15% by weight, and most preferably about 65% to about 20% by weight of the viscous gel. The viscous gel formed by mixing the polymer and the solvent typically exhibits a viscosity of from about 100 to about 200,000 poise, preferably from about 500 to about 50,000 poise, often from about 1,000 to about 50,000 poise measured at a 1 sec⁻¹ shear rate and 25° C. using a Haake Rheometer at about 1-2 days after mixing is completed. Mixing the polymer with the solvent can be achieved with conventional low shear equipment such as a Ross double planetary mixer for from about 10 minutes to about 1 hour, although shorter and longer periods may be chosen by one skilled in the art depending on the particular physical characteristics of the composition being prepared. Since it is often desirable to administer the implant as an injectable composition, a countervailing consideration when forming implants that are viscous gels is that the polymer/solvent/beneficial agent composition have sufficiently low viscosity in order to permit it to be forced through a small diameter, e.g., 16 gauge and higher, preferably 20 gauge and higher, more preferably 22 gauge and higher, even more preferably 24 gauge and higher gauge needle. If necessary, adjustment of viscosity of the gel for injection can be accomplished with emulsifying agents as described herein. Yet, such compositions should have adequate dimensional stability so as to remain localized and be able to be removed if necessary. The particular gel or gel-like compositions of the present invention satisfy such requirements.

Beneficial Agents:

The beneficial agent can be any physiologically or pharmacologically active substance or substances optionally in combination with pharmaceutically acceptable carriers and additional ingredients such as antioxidants, stabilizing agents, permeation enhancers, etc. that do not substantially adversely affect the advantageous results that can be attained by the present invention. The beneficial agent may be any of the agents which are known to be delivered to the body of a human or an animal and that are preferentially soluble in water rather than in the polymer-dissolving solvent. These agents include drug agents, medicaments, vitamins, nutrients, or the like. Included among the types of agents which meet this description are lower molecular weight compounds, proteins, peptides, genetic material, nutrients, vitamins, food supplements, sex sterilants, fertility inhibitors and fertility promoters.

Beneficial agents (drugs) which may be delivered by the present invention include drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, a drug, proteins, enzymes, hormones, polynucleotides, nucleoproteins, polysaccharides, glycoproteins, lipoproteins, polypeptides, steroids, analgesics, local anesthetics, antibiotic agents, chemotherapeutic agents, immunosuppressive agents, anti-inflammatory agents including anti-inflammatory corticosteroids, antiproliferative agents, antimitotic agents, angiogenic agents, anticoagulants, fibrinolytic agents, growth factors, antibodies, ocular drugs, and metabolites, analogs (including synthetic and substituted analogs), derivatives (including aggregative conjugates/fusion with other macromolecules and covalent conjugates with unrelated chemical moieties by means known in the art) fragments, and purified, isolated, recombinant and chemically synthesized versions of these species.

Examples of drugs that may be delivered by the composition of the present invention include, but are not limited to, procaine, procaine hydrochloride, tetracaine, tetracaine hydrochloride, cocaine, cocaine hydrochloride, chloroprocaine, chloroprocaine hydrochloride, proparacaine, proparacaine hydrochloride, piperocaine, piperocaine hydrochloride, hexylcaine, hexylcaine hydrochloride, naepaine, naepaine hydrochloride, benzoxinate, benzoxinate hydrochloride, cyclomethylcaine, cyclomethylcaine hydrochloride, cyclomethylcaine sulfate, lidocaine, lidocaine hydrochloride, bupivicaine, bupivicaine hydrochloride, mepivicaine, mepivacaine hydrochloride, prilocaine, prilocaine hydrochloride, dibucaine and dibucaine hydrochloride, etidocaine, benzocaine, propoxycaine, dyclonin, pramoxine, oxybuprocaine, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17α-hydroxyprogesterone acetate, 19-nor-progesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat, captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth factors such as epidermal growth factors (EGF), platelet-derived growth factors (PDGF), fibroblast growth factors (FGF), transforming growth factors-α (TGF-α), transforming growth factors-β (TGF-β), erythropoietin (EPO), insulin-like growth factor-I (IGF-I), insulin-like growth factor-II (IGF-II), interleukin-1, interleukin-2, interleukin-6, interleukin-8, tumor necrosis factor-α (TNF-α), tumor necrosis factor-β (TNF-β), Interferon-α (INF-α), Interferon-β (INF-β), Interferon-γ (INF-γ), Interferon-ω (INF-ω), colony stimulating factors (CGF), vascular cell growth factor (VEGF), thrombopoietin (TPO), stromal cell-derived factors (SDF), placenta growth factor (PIGF), hepatocyte growth factor (HGF), granulocyte macrophage colony stimulating factor (GM-CSF), glial-derived neurotropin factor (GDNF), granulocyte colony stimulating factor (G-CSF), ciliary neurotropic factor (CNTF), bone morphogeneic proteins (BMP), coagulation factors, human pancreas hormone releasing factor, analogs and derivatives of these compounds, and pharmaceutically acceptable salts of these compounds, or their analogs or derivatives.

Additional examples of drugs that may be delivered by the composition of the present invention include, but are not limited to, antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin, actinomycin D, daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP)II_{b}IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine (cladribine)); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); antiinflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen); indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blocker; nitric oxide donors; anti-sense oligionucleotides and combinations thereof; cell cycle inhibitors, mTOR inhibitors, and growth factor signal transduction kinase inhibitors, analogs and derivatives of these compounds, and pharmaceutically acceptable salts of these compounds, or their analogs or derivatives.

In certain preferred embodiments, the beneficial agent includes chemotactic growth factors, proliferative growth factors, stimulatory growth factors, and transformational peptide growth factors including genes, precursors, post-translational-variants, metabolites, binding-proteins, receptors, receptor agonists and antagonists of the following growth factor families: epidermal growth factors (EGFs), platelet-derived growth factor (PDGFs), insulin-like growth factors (IGFs), fibroblast-growth factors (FGFs), transforming-growth factors (TGFs), interleukins (ILs), colony-stimulating factors (CSFs, MCFs, GCSFs, GMCSFs), Interferons (IFNs), endothelial growth factors (VEGF, EGFs), erythropoietins (EPOs), angiopoietins (ANGs), placenta-derived growth factors (PIGFs), and hypoxia induced transcriptional regulators (HIFs).

The present invention also finds application with chemotherapeutic agents for the local application of such agents to avoid or minimize systemic side effects. Gels of the present invention containing chemotherapeutic agents may be injected directly into the tumor tissue for sustained delivery of the chemotherapeutic agent over time. In some cases, particularly after resection of the tumor, the gel may be implanted directly into the resulting cavity or may be applied to the remaining tissue as a coating. In cases in which the gel is implanted after surgery, it is possible to utilize gels having higher viscosities since they do not have to pass through a small diameter needle. Representative chemotherapeutic agents that may be delivered in accordance with the practice of the present invention include, for example, carboplatin, cisplatin, paclitaxel, BCNU, vincristine, camptothecin, etopside, cytokines, ribozymes, interferons, oligonucleotides and oligonucleotide sequences that inhibit translation or transcription of tumor genes, functional derivatives of the foregoing, and generally known chemotherapeutic agents such as those described in U.S. Pat. No. 5,651,986. The present application has particular utility in the sustained delivery of water soluble chemotherapeutic agents, such as for example cisplatin and carboplatin and the water soluble derivatives of paclitaxel. Those characteristics of the invention that minimize the burst effect are particularly advantageous in the administration of water soluble beneficial agents of all kinds, but particularly those compounds that are clinically useful and effective but may have adverse side effects.

To the extent not mentioned above, the beneficial agents described in aforementioned U.S. Pat. No. 5,242,910 can also be used. One particular advantage of the present invention is that materials, such as proteins, as exemplified by the enzyme lysozyme, and cDNA, and DNA incorporated into vectors both viral and nonviral, which are difficult to microencapsulate or process into microspheres can be incorporated into the compositions of the present invention without the level of degradation caused by exposure to high temperatures and denaturing solvents often present in other processing techniques.

The particles with beneficial agent that are incorporated into the viscous gel formed from the polymer and the solvent typically have an average particle size of from about 0.1 to about 250 microns, preferably from about 1 to about 200 microns and often from 30 to 125 microns. For instance, particles having an average particle size of about 5 microns have been produced by spray drying or freeze drying an aqueous mixture containing 50% sucrose and 50% chicken lysozyme (on a dry weight basis) and mixtures of 10-20% hGH and 15-30 mM zinc acetate. Other examples are described below. Conventional lyophilization processes can also be utilized to form particles of beneficial agents of varying sizes using appropriate freezing and drying cycles. The particle size can also be controlled by separating out the desired ranges, e.g., by sieving.

To form a suspension or dispersion of particles of the beneficial agent in the viscous gel formed from the polymer and the solvent, any conventional low shear device can be used such as a Ross double planetary mixer at ambient conditions. In this manner, efficient distribution of the particles with the beneficial agent can be achieved substantially without degrading the beneficial agent.

The beneficial agent can typically be dissolved or dispersed, or held in particulates in the composition in an amount of from about 0.1% to about 50% by weight, preferably in an amount of from about 1% to about 40%, more preferably in an amount of about 2% to about 30%, and often 2 to 20% by weight of the combined amounts of the polymer, solvent, release rate controlling agent, excipients, and beneficial agent. Depending on the amount of beneficial agent present in the composition, one can obtain different release profiles and burst indices. More specifically, for a given polymer and solvent, by adjusting the amounts of these components and the amount of the beneficial agent, one can obtain a release profile that depends more on the degradation of the polymer than the diffusion of the beneficial agent from the composition or vice versa. In this respect, at lower beneficial agent loading rates, one generally obtains a release profile reflecting degradation of the polymer wherein the release rate increases with time. At higher loading rates, one generally obtains a release profile caused by diffusion of the beneficial agent wherein the release rate decreases with time. At intermediate loading rates, one obtains combined release profiles so that if desired, a substantially constant release rate can be attained. In order to minimize burst, loading of beneficial agent on the order of 30% or less by weight of the overall gel composition, i.e., polymer, solvent and beneficial agent, is preferred, and loading of 20% or less is more preferred.

Release rates and loading of beneficial agent will be adjusted to provide for therapeutically effective delivery of the beneficial agent over the intended sustained delivery period. As already mentioned, the beneficial agent is preferably held in particulates dispersed in the depot composition. The beneficial agent can also be present in the polymer gel at concentrations that are above the saturation concentration of beneficial agent in water to provide a drug reservoir from which the beneficial agent is dispensed. While the release rate of beneficial agent depends on the particular circumstances, such as the beneficial agent to be administered, release rates on the order of from about 0.1 micrograms/day to about 30 milligrams/day, preferably from about 1 microgram/day to about 20 milligrams per day, more preferably from about 10 micrograms/day to about 10 milligram/day, for periods of from about 24 hours to about 180 days, preferably 24 hours to about 120 days, more preferably 24 hours to about 90 days, often 3 days to about 90 days can be obtained. Further, the dose of beneficial agent may be adjusted by adjusting the amount of depot gel injected. Greater amounts may be delivered if delivery is to occur over shorter periods. Generally, higher release rate is possible if a greater burst can be tolerated. In instances where the gel composition is surgically implanted, or used as a “leave behind” depot when surgery to treat the disease state or another condition is concurrently conducted, it is possible to provide higher doses that would normally be administered if the implant was injected. Further, the dose of beneficial agent may be controlled by adjusting the volume of the gel implanted or the injectable gel injected.

Further, the release rate is also controlled by the nature of the particles and the particle loading in the depot gel.

Preferably, the system releases 40% or less by weight of the beneficial agent present in the viscous gel within the first 24 hours after implantation in the subject. More preferably, 30% or less by weight of the beneficial agent will be released within the first 24 hours after implantation, and the implanted composition has a burst index of 12 or less, preferably 8 or less.

Optional Additional Components:

Other components may be present in the gel composition, to the extent they are desired or provide useful properties to the composition, such as polyethylene glycol, hydroscopic agents, stabilizing agents (for example surfactants like Tween 20, Tween 80, and the like, sugars such as sucrose, treholose, and the like, salts, antioxidants), pore forming agents, bulking agents (such as sorbitol, mannitol, glycine, and the like), chelating agents (such as divalent metal ions including zinc, magnesium, calcium, copper and the like), buffering agents (such as phosphate, acetane, succinate, histidine, TRIS, and the like) and others. When the composition includes a peptide or a protein that is soluble in or unstable in an aqueous environment, it may be highly desirable to include a solubility modulator that may, for example, be a stabilizing agent, in the composition. Various modulating agents are described in U.S. Pat. Nos. 5,654,010 and 5,656,297, the disclosures of which are incorporated herein by reference. In the case of hGH, for example, it is preferable to include an amount of a salt of a divalent metal, preferably zinc. Examples of such modulators and stabilizing agents, which may form complexes with the beneficial agent or associate to provide the stabilizing or modulated release effect, include metal cations, preferably divalent, present in the composition as magnesium carbonate, zinc carbonate, calcium carbonate, magnesium acetate, magnesium sulfate, zinc acetate, zinc sulfate, zinc chloride, magnesium chloride, magnesium oxide, magnesium hydroxide, other antacids, and the like. The amounts of such agents used will depend on the nature of the complex formed, if any, or the nature of the association between the beneficial agent and the agent. Molar ratios of solubility modulator or stabilizing agent to beneficial agent of about 100:1 to 1:1, preferably 10:1 to 1:1, typically can be utilized.

Pore forming agents include biocompatible materials that when contacted with body fluids dissolve, disperse or degrade to create pores or channels in the polymer matrix. Typically, organic and non-organic materials that are water soluble such as sugars (e.g., sucrose, dextrose), water soluble salts (e.g., sodium chloride, sodium phosphate, potassium chloride, and sodium carbonate), water soluble solvents such as N-methyl-2-pyrrolidone and polyethylene glycol and water soluble polymers (e.g., carboxymethylcellulose, hydroxypropyl-cellulose, and the like) can conveniently be used as pore formers. Such materials may be present in amounts varying from about 0.1% to about 100% of the weight of the polymer, but will typically be less than 50% and more typically less than 10-20% of the weight of polymer.

Utility and Administration:

The means of administration of the implants is not limited to injection, although that mode of delivery may often be preferred. Where the implant will be administered as a leave-behind product, it may be formed to fit into a body cavity existing after completion of surgery or it may be applied as a flowable gel by brushing or palleting the gel onto residual tissue or bone. Such applications may permit loading of beneficial agent in the gel above concentrations typically present with injectable compositions.

To further understand the various aspects of the present invention, the results set forth in the previously described figures were obtained in accordance with the following examples.

### EXAMPLES

### Example 1

### Depot Gel Vehicle Preparation

A gel vehicle for use in an injectable depot of the composition was prepared as follows. A glass vessel was tared on a Mettler PJ3000 top loader balance. Poly (D,L-lactide-co-glycolide) (PLGA), available as 50:50 Resomer® RG502 (PLGA RG 502), was weighed into the glass vessel. The glass vessel containing PLGA was tared and the corresponding solvent was added. Amounts expressed as percentages for various polymer/solvent combinations are set forth in Table 1, below. The polymer/solvent mixture was manually stirred with a stainless steel square-tip spatula, resulting in a sticky amber paste-like substance containing white polymer particles. The vessel containing the polymer/solvent mixture was sealed and placed in a temperature controlled incubator equilibrated to 39° C. The polymer/solvent mixture was removed from the incubator when it appeared to be a clear amber homogeneous gel. Incubation time intervals ranged from 1 to 4 days, depending on solvent and polymer type and solvent and polymer ratios. Thereafter, the mixture was placed in an oven (65° C.) for 30 minutes. It was noted that the PLGA-504 was dissolved in the mixture upon removal from the oven.

Additional depot gel vehicles are prepared with the following solvents or mixtures: benzyl benzoate (“BB”), benzyl alcohol (“BA”), and propylene glycol (“PG”), and the following polymers: Poly (D,L-lactide) Resomer® L104, PLA-L104, code no. 33007, Poly (D,L-lactide-co-glycolide) 50:50 Resomer® RG502, code 0000366, Poly (D,L-lactide-co-glycolide) 50:50 Resomer® RG502H, PLGA-502H, code no. 260187, Poly (D,L-lactide-co-glycolide) 50:50 Resomer® RG503, PLGA-503, code no. 0080765, Poly (D,L-lactide-co-glycolide) 50:50 Resomer® RG755, PLGA-755, code no. 95037, Poly L-Lactide MW 2,000 (Resomer® L 206, Resomer® L 207, Resomer® L 209, Resomer® L 214); Poly D,L Lactide (Resomer® R 104, Resomer® R 202, Resomer® R 203, Resomer® R 206, Resomer® R 207, Resomer® R 208); Poly L-Lactide-co-D,L-lactide 90:10 (Resomer® LR 209); Poly D-L-lactide-co-glycolide 75:25 (Resomer® RG 752, Resomer® RG 756); Poly D,L-lactide-co-glycolide 85:15 (Resomer® RG 858); Poly L-lactide-co-trimethylene carbonate 71:30 (Resomer® LT 706); Poly dioxanone (Resomer® X 210) (Boehringer Ingelheim Chemicals, Inc., Petersburg, Va.); DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 MEDISORB® Polymer 5050 DL 2A(3), MEDISORB® Polymer 5050 DL 3A(3), MEDISORB® Polymer 5050 DL 4A(3)) (Medisorb Technologies International L.P., Cincinatti, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ε-caprolactone; Poly DL-lactide-co-caprolactone 25:75; and Poly DL-lactide-co-caprolactone 75:25 (Birmingham Polymers, Inc., Birmingham, Ala.). Typical polymer molecular weights were in the range of 14,400-39,700 (M_{w}) [6,400-12,000 (Mₙ)]. Representative gel vehicles are described in Table 1 below.

TABLE 1

| | PLGA | Benzyl | Benzyl | |
|---|---|---|---|---|
| | RG502 | Benzoate | Alcohol | PG |
| Formulation | (g) | (g) | (g) | (g) |
| 1 | 5.0365 | 4.5093 | 0.5178 | — |
| 2 | 5.0139 | 3.7553 | 1.2560 | — |
| 3 | 5.0350 | 4.5193 | — | 0.5206 |
| 4 | 5.0024 | 3.7547 | — | 1.2508 |
| 5 | 5.0068 | 5.0044 | — | — |

### Example 2

### Viscosity Measurements on Depot Gel Vehicles

Rheological behavior was tested for depot vehicles formulated with different solvents. A vehicle comprising 50 wt. % polymer (PLGA RG502) and 50 wt. % solvent (benzyl alcohol) was prepared according to the procedures outlined in Example 1. For comparative purposes, solvent containing benzyl benzoate (formulation 5) and solvent containing benzyl benzoate combined with ethanol (formulation 6) were also prepared. Table 2 lists the formulations used in the test.

TABLE 2

| | | Benzyl | Benzyl Alcohol | Ethanol |
|---|---|---|---|---|
| Formulation | Polymer (%) | Benzoate (%) | (%) | (%) |
| 5 | 50.0 | 50.0 | 0.0 | 0.0 |
| 6 | 45.0 | 52.8 | 0.0 | 2.2 |
| 7 | 50.0 | 0.0 | 50.0 | 0.0 |

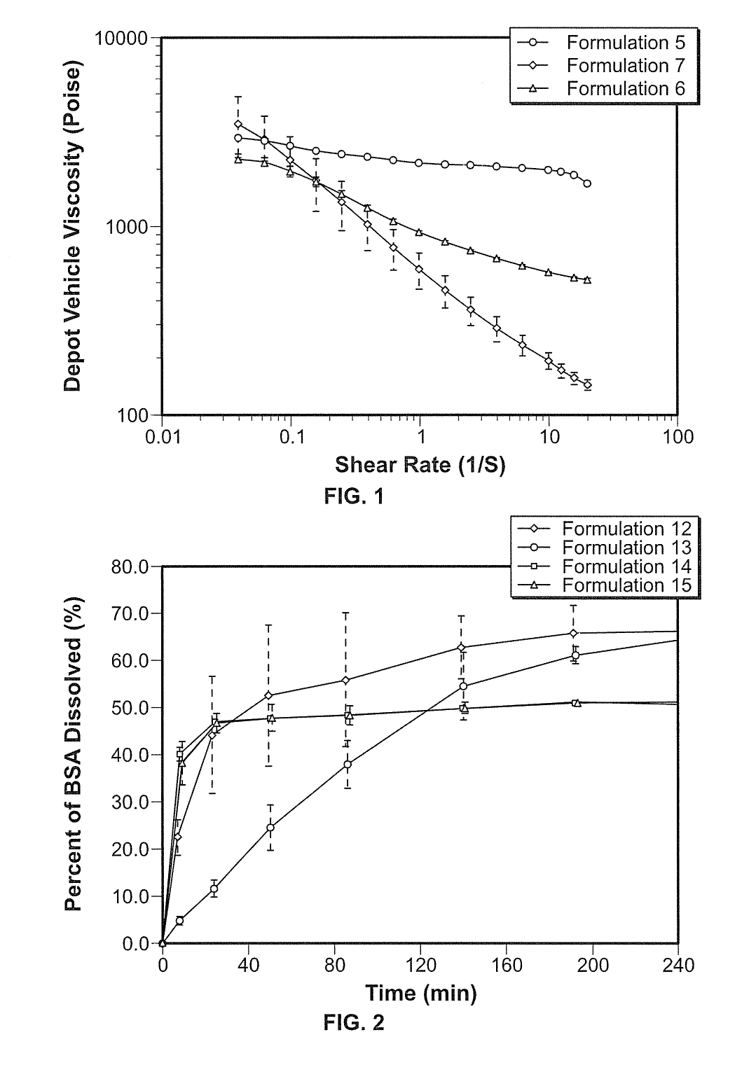

Formulations 5, 6 and 7 were tested for viscosity under various shear rates. As indicated in FIG. 1, significant shear thinning behavior was observed when benzyl alcohol was used as the solvent (formulation 7), in contrast to formulations using benzyl benzoate (formulation 5) and benzyl benzoate with ethanol (formulation 6) as a co-solvent, respectively.

### Example 3

### HGH Particle Preparation

Human growth hormone (hGH) particles were prepared as follows:

hGH solution (5 mg/ml) solution in water (BresaGen Corporation, Adelaide, Australia) was concentrated to 10 mg/mL using a Concentration/Dialysis Selector diafiltering apparatus. The diafiltered hGH solution was washed with 5 times volume of tris or phosphate buffer solution (pH 7.6). Particles of hGH were then formed by spray drying or lyophilization using conventional techniques either spray drying or lyophilization followed by grinding and sieving. Phosphate buffer solutions (5 or 50 mM) containing hGH (5 mg/mL) were spray dried. Zinc complex particles were prepared in TRIS buffer and formed in a similar way with various levels of zinc acetate (0 to 30 mM). Other zinc complex particles are shown in Example 7 below. The spray-dried particles were produced using a Yamato Mini Spray dryer set at the following parameters:

| Spray Dryer Parameter | Setting |
|---|---|
| Atomizing Air | 2 psi |
| Inlet Temperature | 120° C. |
| Aspirator Dial | 7.5 |
| Solution Pump | 2-4 |
| Main Air Valve | 40-45 psi |

hGH particles having a size range between 2-100 microns were obtained.

Lyophilized particles were prepared from tris buffer solutions (5 or 50 mM: pH 7.6) containing hGH (5 mg/mL) using a Durastop Lyophilizer in accordance with the following freezing and drying cycles:

| | |
|---|---|
| Freezing | Ramp down at 2.5 C./min to −30° C. and hold for 30 min |
| cycle | Ramp down at 2.5 C./min to −30° C. and hold for 30 min |
| Drying | Ramp up at 0.5 C./min to 10° C. and hold for 960 min |
| cycle | Ramp up at 0.5 C./min to 20° C. and hold for 480 min |
| | Ramp up at 0.5 C./min to 25° C. and hold for 300 min |
| | Ramp up at 0.5 C./min to 30° C. and hold for 300 min |
| | Ramp up at 0.5 C./min to 5° C. and hold for 5000 min |

The lyophilized hGH formulations were ground and sieved through a 70 mesh screen followed by a 400 mesh screen to obtain particles having a size range between 38-212 microns.

### Example 4

### HGH-Stearic Acid Particle Preparation

Human growth hormone (hGH) particles were prepared as follows: Lyophilized hGH (3.22 grams, Pharmacia-Upjohn, Stockholm, Sweden) and stearic acid (3.22 grams, 95% pure, Sigma-Aldrich Corporation, St. Louis, Mo.) were blended and ground. The ground material was compressed in a 13 mm round die, with a force of 10,000 pounds for 5 minutes. Compressed tablets were ground and sieved through a 70 mesh screen followed by a 400 mesh screen to obtain particles having a size range between 38-212 microns.

### Example 5

### HGH-Pluronic Particle Preparation

Two different methods were used to prepare the hGH/Pluronic particles (Table 3):
1) Solution of hGH in 5 mM, pH 7.8 phosphate buffer were mixed with 2% of Pluronic F127 (BASF, USA) in 5 mM, pH 7.8 phosphate buffer with the final ratios of hGH/Pluronic 5:5 and 7:3 by wt, respectively. (Pluronic F127 gel is a thermal reversible substance.) The resulting solutions were then lyophilized. After lyophilization, the particles were ground in a mortar and pestle and sieved to between 38 μm and 212 μm.

2) Solution of hGH in 5 mM, pH 7.8 phosphate buffer were mixed with 2% of Pluronic F127 (BASF, USA) in 5 mM, pH 7.8 phosphate buffer with the final ratios of hGH/Pluronic 5:5 and 7:3 by wt, respectively. The resulting solutions were then spray dried according to the conditions described in Example 3.

TABLE 3

| | hGH | | |
|---|---|---|---|
| Formulation | (%) | Pluronic F127 (%) | Process |
| 8 | 50.0 | 50.0 | Lyophilized |
| 9 | 70.0 | 30.0 | Lyophilized |
| 10 | 50.0 | 50.0 | Spray dried |
| 11 | 70.0 | 30.0 | Spray dried |

### Example 6

### Protein/Alginate Particle Preparation

Sodium alginate can form ionically crosslinked networks with some bivalent cationic ions such as calcium. When formulated with alginate/Ca, drugs (e.g. proteins) can be trapped in the crosslinked network and be slowly released out of network through diffusion and/or ionic exchange (breaking the network). The drug formulation can be loaded into the injectable depot of this invention. The release of the drug (e.g. protein) from the depot will be dually controlled by both drug formulation (through diffusion from the particles) and the depot matrix.

Bovine Serum Albumin (BSA, Sigma, St Luise, Mo., USA), sodium alginate (Pronova, Demark) and calcium lactate (Sigma, St Luise, Mo., USA) were dissolved in 5 mM TRIS buffer, pH 7.0. The ratio of BSA/Alginate/Calcium lactate can be varied. Examples of formulations are listed in Table 3. The solution of three components was lyophilized. The lyophilized BSA formulations were ground and sieved through a 70 mesh screen followed by a 400 mesh screen to obtain particles having a size range between 38-212 microns. Furthermore, the solution of three components can also be spray dried according to the conditions described in Example 3.

Similarly, hGH instead of BSA was formulated with alginate as described above.

TABLE 4

| | BSA | Na Alginate | Ca Lactate |
|---|---|---|---|
| Formulation | (%) | (%) | (%) |
| 12 | 50.0 | 50.0 | 0.0 |
| 13 | 50.0 | 47.5 | 2.5 |
| 14 | 50.0 | 45.0 | 5.0 |
| 15 | 50.0 | 42.5 | 7.5 |

### Example 7

### HGH/ZN Complex Particle Preparation

hGH solutions of 40 mg/mL and zinc acetate of 27.2 mM were prepared in 5 mM TRIS buffer, pH 7.0, respectively. A 15:1 final zinc:hGH mole ratio was obtain by mixing equal parts of hGh and zinc acetate solutions together. This solution will be allowed to complex for approximately one hour at 4° C. This complex will be pre-cooled to −70° C. and lyophilized using lyophilization conditions as described in Example 3 above.

Three different hGH/Zn complex particles were prepared as described below (Table 5):
1) The lyophilized hGH/Zn complex was ground using a Waring blender. Particles were collected between a 120-mesh (125 μm) and 400-mesh (38 μm) sieve (Formulation 16).
2) The lyophilized hGH/Zn complex was transferred to a 13 mm round compression die and compressed at 5 tons for 5 minutes to form a pellet. The pellet was ground using a Waring blender. Particles were collected between a 120-mesh (125 μm) and 400-mesh (38 μm) sieve (Formulation 17).

3) In some cases, the lyophilized hGH/Zn complex was mixed with stearic acid in a 1:1 (w/w) ratio. The mixture was compressed into a pellet using a 13 mm compression die under 5 tons of force for 5 minutes. The pellet was ground using a Waring blender. Particles were collected between a 120-mesh (125 μm) and 400-mesh (38 μm) sieve (Formulation 18).

TABLE 5

| | hGH/Zn complex | Stearic acid | |
|---|---|---|---|
| Formulation | (%) | (%) | Process conditions |
| 16 | 100.0 | 0 | Grind and sieve |
| 17 | 100.0 | 0 | Compress, then Grind |
| | | | and sieve |
| 18 | 50.0 | 50.0 | Compress, then Grind |
| | | | and sieve |
| 19 | 100* | 0 | Compress, then Grind |
| | | | and sieve |

*hGH not complex with Zn.

### Example 8

### Protein Particle Dissolution Tests

The dissolution rate of formulated protein particles was determined by placing the particles (0.2-0.5 g) into wire mesh baskets in a VanKel dissolution bath. They were run at 37° C. in 200 ml of PBS. 1.5 ml of samples were taken at predetermined time points and analyzed for protein concentration by UV at 280 nm.

FIG. 2 showed the BSA released from formulation in alginate with or without calcium. The BSA release rate was retarded by the addition of calcium in the formulation to form ionically crosslinked network and the BSA release from the formulations was controlled by diffusion and ionic exchange. The higher calcium lactate loading (e.g., formulation 14 (having 5 wt % calcium lactate) and formulation 15 (having 7.5 wt % calcium lactate) resulted in a smaller percentage of BSA dissolved after about 2 hours. The difference in dissolution rate of the BSA from the particulates shows the effect of the difference in the amount of alginate crosslinking, i.e., higher crosslinking slows down the long term release rate of BSA from the alginate network. This suggests that the protein formulation itself provides one control mechanism to the drug release when combined in the injectable depot formulations.

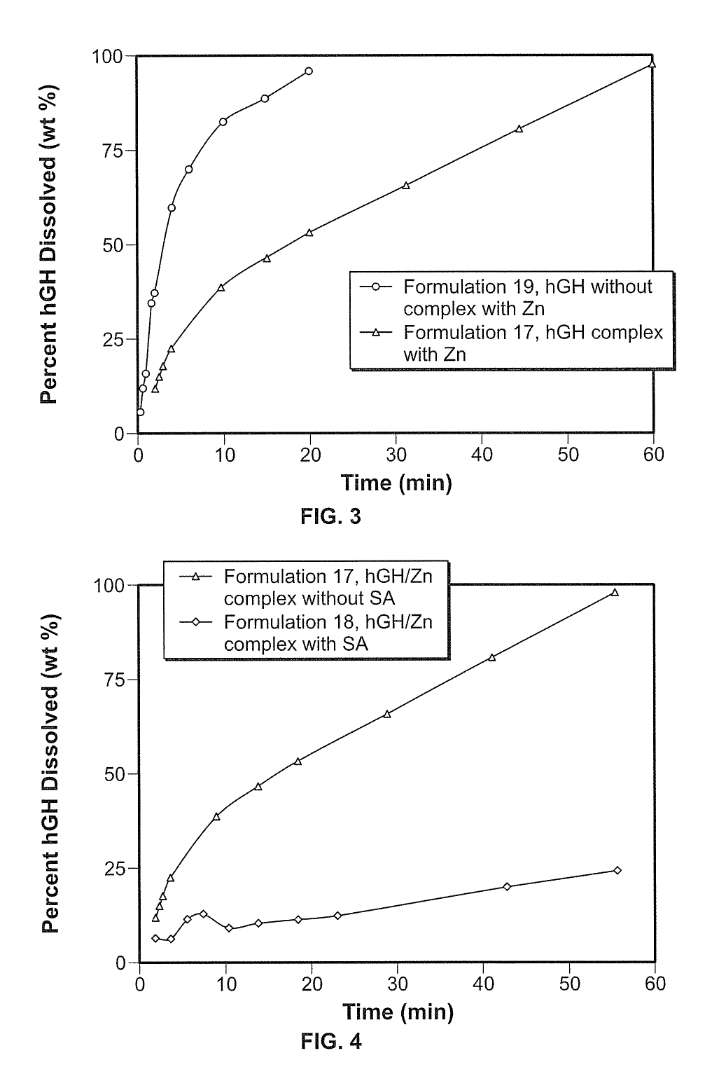

FIGS. 3 & 4 exhibited the dissolution rate of hGH from the formulations with (Formulation 17) or without complex with Zn (Formulation 19), or hGH/Zn complex further mixed with hydrophobic excipient such as stearic acid (SA) (formulation 18). The dissolution rate of hGH was greatly decreased by complex with Zn (FIG. 3). The hGH dissolution rate was further reduced by mixing the hGH/Zn complex with a hydrophobic excipient such as stearic acid (FIG. 4). This indicates that the drug formulation itself provides one control mechanism to the drug release when combined in the injectable depot formulations.

The protein particle dissolution rate studies showed that alginate, stearic acid, zinc complexing, and thermal reversible material each retard the dissolution of protein from the particles. Further, stearic acid when used with zinc complexing further retards protein dissolution rate more than zinc complexing alone. It is expected that when such particles are dispersed in a depot vehicle, the release of protein from the depot after implantation will be affected in a similar way, i.e., in the reduction of initial release rate.

### Example 9

### Bupivacaine-Stearic Acid Particle Preparation

Bupivacaine particles were prepared as follows: Bupivacaine hydrochloride (100 grams, Sigma-Aldrich Corporation, St. Louis, Mo.) particles were sieved through 63-125 micron sieves. The bupivacaine particles and stearic acid (100 grams, 95% pure, Sigma-Aldrich Corporation, St. Louis, Mo.) were blended and ground. The ground material was compressed in a 13 mm round die, with a force of 5,000 pounds for 5 minutes. Compressed tablets were ground and sieved through a 120 mesh screen followed by a 230 mesh screen to obtain particles having a size range between 63-125 microns.

### Example 10

### Drug Loading

Formulated drug particles prepared as described in Examples 3, 4, 5, 6, 7 and 9, were loaded to the depot gel vehicles, as described in Example 1, in an amount of 10-20% by weight and blended manually until the dry powder is wetted completely. Then, the milky light yellow particle/gel mixture was thoroughly blended by conventional mixing using a Caframo mechanical stirrer with an attached square-tip metal spatula. Resulting formulations are illustrated in Table 6 below. Final homogenous depot formulations were transferred to 3, 10 or 30 cc disposable syringes for storage or dispensing.

TABLE 6

| | | Benzyl | Benzyl | Ethanol |
|---|---|---|---|---|
| Formulation | Polymer (%) | Benzoate (%) | Alcohol (%) | (%) |
| 20^{a} | 45.0¹ | 45.0 | 0.0 | 0.0 |
| 21^{a} | 58.5¹ | 31.5 | 0.0 | 0.0 |
| 22^{b} | 58.5¹ | 0.0 | 31.5 | 0.0 |
| 23^{c} | 45.0² | 33.8 | 11.3 | 0.0 |
| 24^{c} | 45.0³ | 33.8 | 11.3 | 0.0 |
| 25^{c} | 39.6² | 49.5 | 0.0 | 0.9 |
| 26^{d} | 39.6² | 49.5 | 0.0 | 0.9 |
| 27^{c} | 45.0² | 45.0 | 0.0 | 0.0 |
| 28^{e} | 45.0² | 45.0 | 0.0 | 0.0 |
| 31^{a} | 67.5⁴ | 0.0 | 22.5 | 0.0 |
| 32^{B} | 60.0⁴ | 0.0 | 20.0 | 0.0 |

¹= PLGA L/G 50/50 (MW 8,000);
²= PLGA L/G 50/50 (MW 16,000);
³= PLGA L/G 50/50 (MW 22,600);
⁴= PLGA L/G 50/50 (MW 10,000);
^{a}= 10% bupivacaine;
^{b}= 5% bupivacaine, 5% SA;
^{B}= 10% bupivacaine, 10% SA;
^{c}= 5% hGH, 5% SA;
^{d}= 10% hGH/Zn complex;
^{e}= 5% hGH, 5% Pluronic F127

### Example 11

### Bupivacaine In Vivo Studies

In vivo studies in rats (n=4-6 per group) were performed following an open protocol to determine plasma levels of bupivacaine upon systemic administration of bupivicaine via the implant systems of this invention. Depot gel bupivacaine formulations (as described in Table 6, Example 10) were loaded into customized 0.5 cc disposable syringes and injected into rats with an 18 Gauge 1 inch needle. Blood was drawn at specified time intervals (1 hour, 4 hours and on days 1, 2, 5, 7, and 9) and analyzed for bupivacaine using LC/MS.

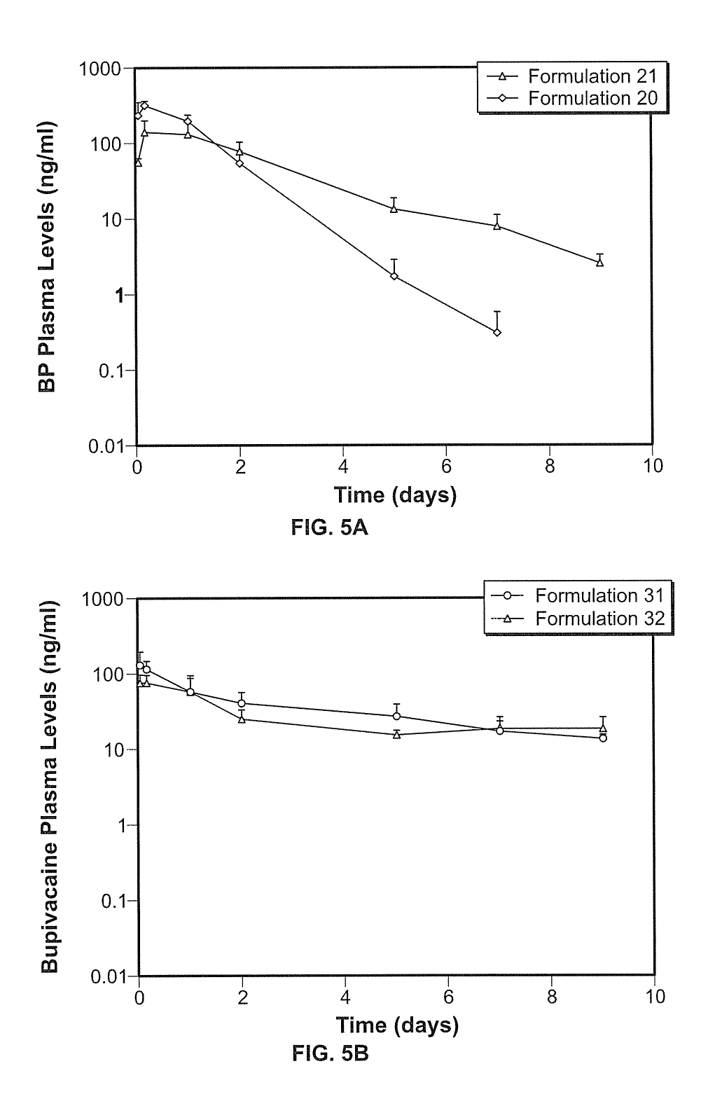

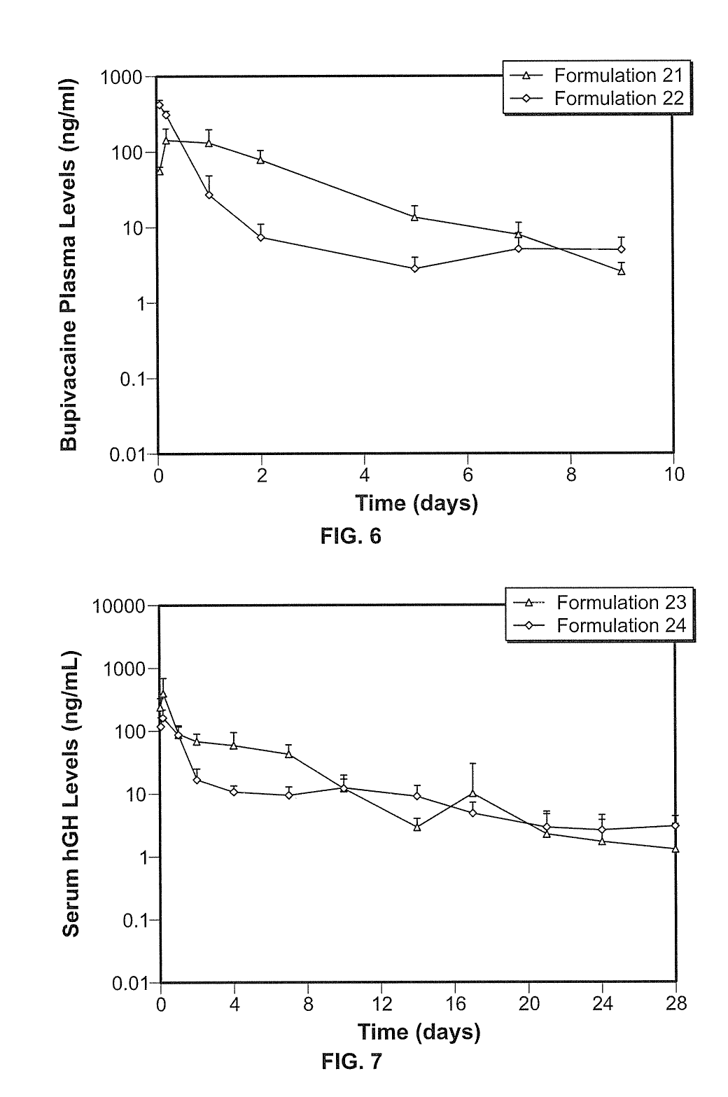

FIGS. 5 & 6 illustrate representative in vivo release profiles of bupivacaine obtained in rats from various depot formulations, including those of the present invention. The in vivo bupivacaine release profile of the depot formulations can be significantly changed by altering the polymer/solvent ratios in the depot vehicle (FIG. 5, formulations 20 vs. 21), indicating that the depot matrix played an important role in controlling the bupivacaine release rate profiles. Formulation 21, having a higher percentage of polymer, had a smaller initial burst and later had a higher plasma drug level than formulation 20. On the other hand, the in vivo bupivacaine release rate profiles were significantly affected by the drug particle formulations (FIG. 6, formulation 21 vs. 22), suggesting that the drug particles in the depot can also play an important role in controlling the overall drug release rate profile. Here, formulation 22 had 5% bupivacaine and 5% stearic acid, yet starting from the second half of day 1, its resulting plasma bupivacaine level was lower and more level (or even) than formulation 21 (which had twice the amount of bupivacaine but no stearic acid) for many days. Further, FIG. 5B (formulations 31 vs. 32) shows that the formulation 32 having 10% stearic acid had a more level plasma bupivacaine concentration than formulation 31 that had the same amount of bupivacaine but no stearic acid. Thus, combination of drug particle formulation with depot vehicle matrix can play dual-roles in controlling the drug release from the depot formulation.

### Example 12

### HGH In Vivo Studies

In vivo studies in rats were performed following an open protocol to determine serum levels of hGH upon systemic administration of hGH via the implant systems of this invention. Depot gel hGH formulations (as described in Table 6, Example 10) were loaded into customized 0.5 cc disposable syringes and injected into rats with an 18 G 1 inch needle. The rats were immune-suppressed with cyclosporine for the whole duration of study and blood was drawn at specified time intervals. All serum samples were stored at 4° C. prior to analysis. Samples were analyzed for intact hGH content using a radio immuno-assay (RIA).

FIGS. 7, 8 and 9 illustrate representative in vivo release profiles of hGH obtained in rats from various depot formulations, including those of the present invention. The in vivo hGH release profile of the depot formulations was significantly changed by altering the molecular weight of polymer used in the depot vehicle (FIG. 7, formulations 23 vs. 24), indicating that the depot matrix played an important role in controlling the hGH release rate profiles. Here, formulation 24 (in which the polymer MW was 22600) had a more even serum hGH level than formulation 23 (in which the polymer MW was lower, i.e., 16000). The rate of decline of serum hGH level was slower in formulation 24, which had a higher molecular weight depot vehicle polymer than formulation 23.

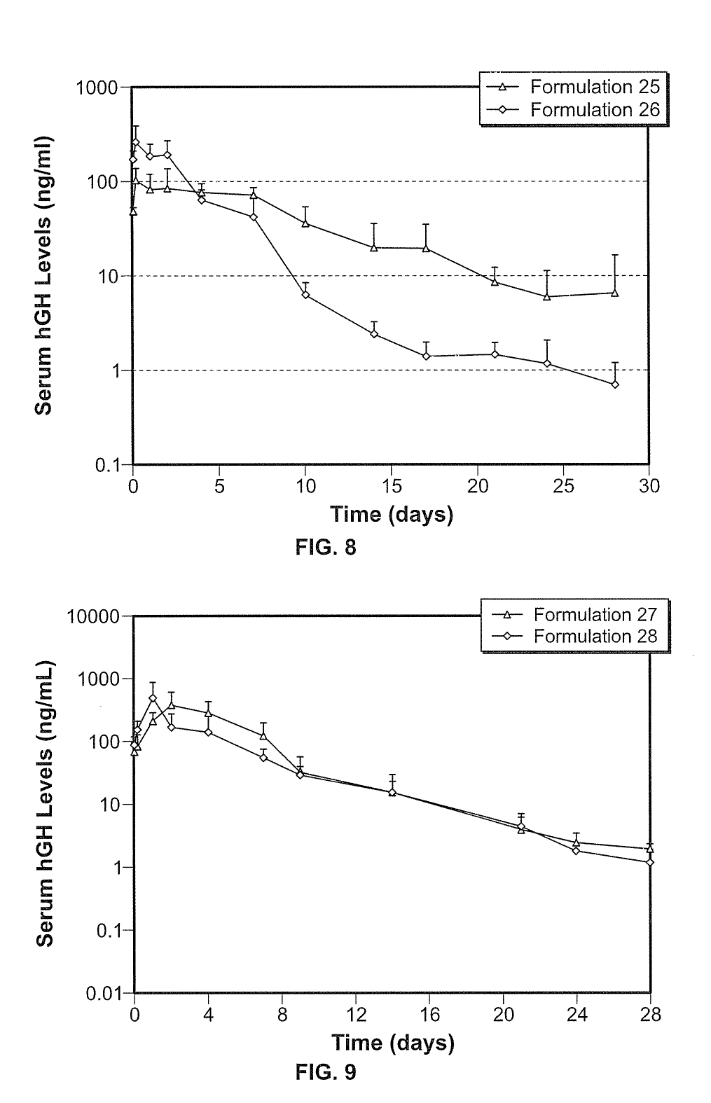

On the other hand, the in vivo hGH release rate profiles can be controlled by the drug particle formulations with different excipients, to create such as hydrophobic microenvironment using fatty acids such as stearic acid (SA), or low solubility metal complex such as zinc, or form thermally reversible gel at body temperature (e.g., using Pluronic F127) so that the drug can be trapped in the gel (FIGS. 8 & 9, formulation 25, 26, 27 & 28).

FIG. 8 compares formulation 25 (which has 5 wt % hGH and 5 wt % stearic acid loaded in the form of particulates) with formulation 26 (which had 10 wt % hGH/Zinc complex loaded in the form of particulates). Formulation 25 that had the stearic acid had a more even serum hGH level than formulation 26 with zinc complex. Further, FIG. 9 compares formulation 27 (which had 5 wt % hGH and 5 wt % stearic acid loaded in the form of particulates) with formulation 28 (which had 5 wt % hGH and 5 wt % of Pluronic F127 loaded in the from of particulates). Formulation 27 that had the stearic acid appeared to have a slightly higher serum hGH level from day 2 to day 8. Thereafter, formulation 27 and 28 had similar serum hGH levels. Thus, Pluronic F127, similar to stearic acid, was effective in reducing initial burst and controlling the release rate if hGH. This suggests that the drug particles in the depot can also play an important role in controlling the overall drug release rate profile. Thus, combination of drug particle formulation with depot vehicle matrix both can play a role in controlling the drug release from the depot formulation.

### Example 13

### Comparison of In Vivo Release of HGH-ZN and HGH

In vivo studies in rats were performed following an open protocol to determine serum levels of hGH upon systemic administration of hGH via the implant systems of this invention. Depot gel hGH-Zn and hGH formulations (as described in Table 7) were loaded into customized 0.5 cc disposable syringes and injected into rats with an 18 G 1 inch needle. The rats were immune-suppressed with cyclosporine for the whole duration of study and blood was drawn at specified time intervals. All serum samples were stored at 4° C. prior to analysis. Samples were analyzed for intact hGH content using a radio immuno-assay (RIA).

TABLE 7

| Formulation* | Polymer (%) | Benzyl Benzoate (%) | Ethanol (%) |
|---|---|---|---|
| 29^{a} | 40.70 | 50.88 | 0.93 |
| 30^{b} | 41.93 | 52.41 | 0.95 |

*Both formulations contain 4.3% hGH active;
^{a}Loaded with lyophilized hGH particles without complex with Zn;
^{b}Loaded with particles of hGH-Zn complex.

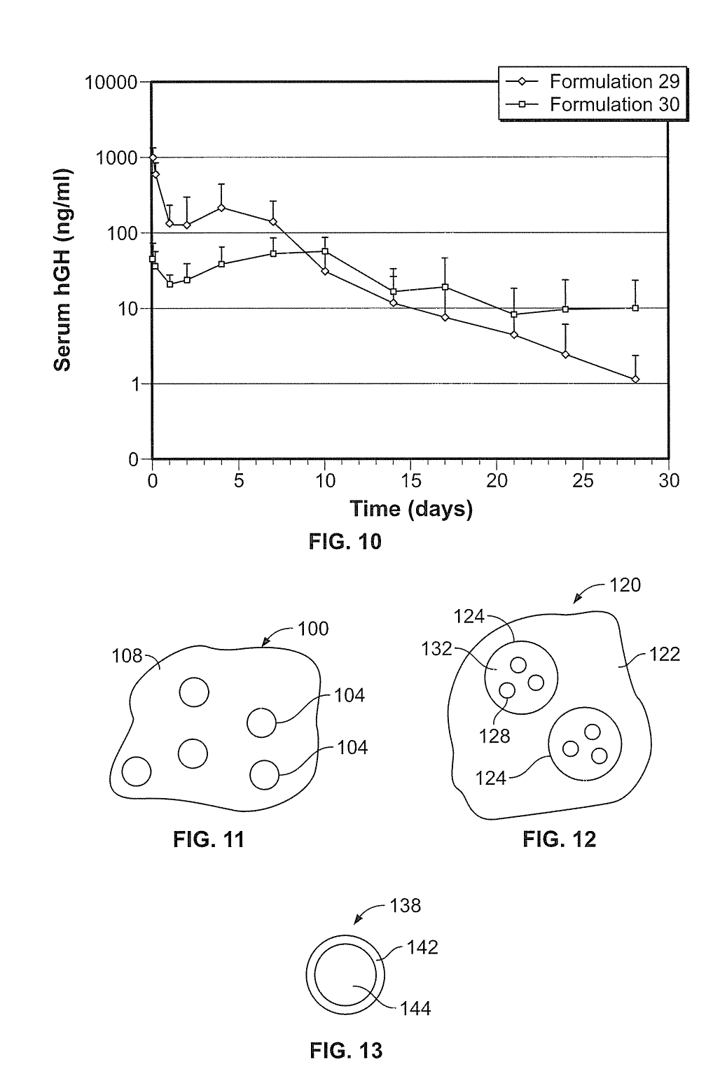

FIG. 10 illustrates the in vivo release profile of hGH and hGH-Zn complex from depot gels. The dual control concept is clearly demonstrated. The release of hGH from formulation 30 was significantly different from that of formulation 29. Formulation 30, which contained zinc complex, shows a much flatter release profile than formulation 29, which did not. The slower dissolution rate of hGH-Zn complex resulted in a slower release of hGH from the depot gel. Therefore, the release profile of hGH is both controlled by the depot gel (the matrix) and the protein particles.

The above-described exemplary embodiments are intended to be illustrative in all respects, rather than restrictive, of the present invention. Thus the present invention is capable of many variations in detailed implementation that can be derived from the description contained herein by a person skilled in the art. All such variations and modifications are considered to be within the scope of the present invention.

What is claimed is:

1. A composition for sustained beneficial agent delivery in a patient, comprising;
a. an injectable depot vehicle containing a bioerodible polymer in an organic solvent, for forming a bioerodible depot implant after administration to a patient;
b. bioerodible particles comprising a beneficial agent and a non-complex release rate controlling agent retarding the release of the beneficial agent from the bioerodible particles; the bioerodible particles being dispersed in the injectable depot vehicle.

2. The composition of claim 1 wherein the non-complex release rate controlling agent hinders movement of the beneficial agent in the bioerodible particles.

3. The composition of claim 2 wherein the non-complex release rate controlling agent functions as at least one of hindering water access by hydrophobicity, being polymeric to form a gel in physiological condition, thermally reversing between ambient condition and physiological condition, having ionic crosslink network which breaks down by ion exchange.

4. The composition of claim 2 wherein the non-complex release rate controlling agent is selected from the group consisting of fatty acid, esters of fatty acid, thermally reversible polymer, and ionic polymer network forming agent.

5. The composition of claim 2 wherein the non-complex release rate controlling agent is selected from the group consisting of fatty acid, esters of fatty acid and thermally reversible gel forming polymer.

6. The composition of claim 2 wherein non-complex release rate controlling agent comprises a fatty acid.

7. The composition of claim 2 wherein non-complex release rate controlling agent comprises an ester of fatty acid.

8. The composition of claim 2 wherein non-complex release rate controlling agent comprises a thermally reversible polymer.

9. The composition of claim 2 wherein non-complex release rate controlling agent is one of two or more release rate controlling agents, a second release rate controlling agent of the two or more release rate controlling agents being selected from the group consisting of fatty acid, ester of fatty acid, thermally reversible polymer, ionic polymer with a network forming agent, and a complex forming agent.

10. The composition of claim 2 wherein there are at least two release rate controlling agents and the bioerodible particles comprising fatty acid and complex forming agent.

11. The composition of claim 1 wherein the non-complex release rate controlling agent is solid in body temperature entrapping the beneficial agent in the bioerodible particles and erodes in vivo.

12. The composition of claim 1 comprising bioerodible subparticles enclosed within the bioerodible particles, the bioerodible subparticles comprising the beneficial agent and a release rate controlling agent.

13. The composition of claim 12 wherein the non-complex release rate controlling agent is one of at least two release rate controlling agents in the bioerodible particle, one of the release rate controlling agents being outside the bioerodible subparticles, another of the release rate controlling agents being inside the bioerodible subparticles and being different from the one outside the bioerodible subparticles.

14. The composition of claim 13 wherein the release rate controlling agent in the subparticles functions in at least one of hindering water access by hydrophobicity, being polymeric to form a gel in physiological condition, complexing with the beneficial agent, thermally reversing between ambient condition and physiological condition, and having ionic crosslink network that breaks down by ion exchange.

15. The composition of claim 13 wherein the release rate controlling agent in the subparticles is selected from the group consisting of fatty acid, thermally reversible polymer, ionic polymer network forming agent, and complex forming agent.

16. The composition of claim 2 wherein the organic solvent is selected from the group consisting of: an aromatic alcohol, lower alkyl esters of aryl acids, lower aralkyl esters of aryl acids; aryl ketones, aralkyl ketones, lower alkyl ketones, lower alkyl esters of citric acid, and combinations thereof.

17. The composition of claim 2 wherein the organic solvent comprises at least one of benzyl alcohol, benzyl benzoate, ethyl benzoate and triacetin.

18. The composition of claim 2 wherein the organic solvent comprises benzyl alcohol and wherein the composition is free of monohydric lower alkanols and free of solvents having miscibility in water that is greater than 7 wt % at 25° C.

19. The composition of claim 2 wherein the bioerodible polymer comprises a lactic acid-based polymer.

20. A method of forming a composition for sustained beneficial agent delivery in a patient, comprising;
a. preparing an injectable depot vehicle containing a bioerodible polymer in an organic solvent, for forming a bioerodible depot implant after administration to the patient;
b. preparing bioerodible particles comprising a beneficial agent and a non-complex release rate controlling agent retarding the release of the beneficial agent from the bioerodible particles; and
c. dispersing the bioerodible beneficial agent particles in the injectable depot vehicle.

21. The method of claim 20 wherein the release rate controlling agent hinders movement of the beneficial agent in the bioerodible particles.

22. The method of claim 21 wherein the non-complex release rate controlling agent functions as at least one of hindering water access by hydrophobicity, being polymeric to form a gel in physiological condition, thermally reversing between ambient condition and physiological condition, having ionic crosslink network which breaks down by ion exchange.

23. The method of claim 21 wherein the non-complex release rate controlling agent is selected from the group consisting of fatty acid, thermally reversible polymer, and gel forming polymer.

24. The method of claim 21 wherein the non-complex release rate controlling agent is selected from the group consisting of fatty acid and thermally reversible gel forming polymer.

25. The method of claim 20 comprising forming the bioerodible particles via a step of forming and compacting particles into larger particles.

26. The method of claim 20 comprising forming the bioerodible particles via a step of forming and compacting particles into larger particles and then reducing the size of the larger particles.

27. The method of claim 20 comprising forming the bioerodible particles via a step of forming pre-compacting particles by spray-drying or lyophilization, compacting the pre-compacting particles into larger particles and then reducing the size of the larger particles by grinding and sieving.

28. The method of claim 20 comprising forming the bioerodible particles via a step of forming pre-compacting particles by spray-drying or lyophilization, compacting the pre-compacting particles into larger particles and then reducing the size of the larger particles by grinding and sieving to achieve particle size of 30 microns to 250 microns.

29. The method of claim 20 comprising forming the bioerodible particles by including subparticles in the bioerodible particles, the bioerodible subparticles comprising the beneficial agent and a release rate controlling agent.

30. The method of claim 20 wherein the non-complex release rate controlling agent is one of at least two release rate controlling agents in the bioerodible particle, one of the release rate controlling agents being outside the bioerodible subparticles, another of the release rate controlling agents being inside the bioerodible subparticles and being different from the one outside the bioerodible subparticles.

31. The method of claim 30 wherein the release rate controlling agent in the subparticles functions in at least one of hindering water access by hydrophobicity, being polymeric to form a gel in physiological condition, complexing with the beneficial agent, thermally reversing between ambient condition and physiological condition, and having ionic crosslink network which breaks down by ion exchange.

32. The method of claim 31 wherein the organic solvent comprises benzyl alcohol and wherein the composition is free of monohydric lower alkanols and free of solvents having miscibility in water that is greater than 7 wt. % at 25° C.

33. A method of administering a beneficial agent to an individual in need thereof, comprising,
a. providing a composition that includes an injectable depot vehicle containing a bioerodible polymer in an organic solvent, for forming a bioerodible depot implant after administration to the individual; and bioerodible particles comprising a beneficial agent and a non-complex release rate controlling agent retarding the release of the beneficial agent from the bioerodible particles; the bioerodible particles being dispersed in the injectable depot vehicle; and
b. administering into the patient the composition.

34. The method of claim 33 wherein the release rate controlling hinders movement of the beneficial agent in the bioerodible particles.

35. The method of claim 34 wherein the non-complex release rate controlling agent functions in at least one of hindering water access by hydrophobicity, being polymeric to form a gel in physiological condition, thermally reversing between ambient condition and physiological condition, having ionic crosslink network which breaks down by ion exchange.

36. The method of claim 33 wherein the organic solvent comprises benzyl alcohol and wherein the composition is free of monohydric lower alkanols and free of solvents having miscibility in water that is greater than 7 wt % at 25° C.

37. The method of claim 33 wherein the bioerodible particles include subparticles in the bioerodible particles, the bioerodible subparticles comprising the beneficial agent and a release rate controlling agent.

38. The method of claim 33 wherein the non-complex release rate controlling agent is one of at least two release rate controlling agents in the bioerodible particle, the bioerodible particle having bioerodible subparticles, one of the release rate controlling agents being outside the bioerodible subparticles, another of the release rate controlling agents being inside the bioerodible subparticles and being different from the one outside the bioerodible subparticles.

39. The method of claim 33 wherein the bioerodible particle having bioerodible subparticles having release rate controlling agent, the release rate controlling agent in the subparticles functions in at least one of hindering water access by hydrophobicity, being polymeric to form a gel in physiological condition, complexing with the beneficial agent, thermally reversing between ambient condition and physiological condition, and having ionic crosslink network which breaks down by ion exchange.

40. A composition for sustained beneficial agent delivery in a patient, comprising;
a. an injectable depot vehicle containing a bioerodible polymer in an organic solvent comprising benzyl alcohol, for forming a bioerodible depot implant after administration to a patient;
b. bioerodible particles comprising a beneficial agent and a non-complex release rate controlling agent retarding the release of the beneficial agent from the bioerodible particles; the bioerodible particles being dispersed in the injectable depot vehicle.

41. A composition for sustained beneficial agent delivery in a patient, comprising;
a. an injectable depot vehicle containing a bioerodible polymer in an organic solvent, for forming a bioerodible depot implant after administration to the patient;
b. bioerodible particles comprising subparticles and a first release rate controlling agent retarding the release of beneficial agent from the bioerodible particles; the bioerodible particles being dispersed in the injectable depot vehicle, the subparticles comprising the beneficial agent and a second release rate controlling agent different from the first release rate controlling agent.
